SEQUENCE LISTING (24 sequences)

SEQ ID NO: 1 - protein, 248 aa, Bombyx mori cytoplasmic polyhedrosis virus
MADVAGTSNRDFRGREQRLFNSEQYNYNNSLNGEVSVWVYAYYSDGSVLVINKNSQYKVGISETFKALKEYREGQHNDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 2 - protein, 245 aa, Autographa californica nucleopolyhedrovirus
MPDYSYRPTIGRTYVYDNKYYKNLDAVIKNAKRKKHFAEHEIEEATLDPLDNYLVAEDPFLGPGKNQKLTLFKEIRNVKPDTMKLVVGWKGKEFYRETWTRFMEDSFPIVNDQEVMDVFLVVNMRPTRPNRCYKFLAQHALRCDPDYVPHDVIRIVEPSWVGSNNEYRISLAKKGGGCPIMNLHSEYTNSFEQFIDRVIWENFYKPIVYIGTDSAEEEEILLEVSLVFKVKEFAPDAPLFTGPAY

SEQ ID NO: 3 - protein, 210 aa, synthetic construct
  REGION 1..210: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 4 - protein, 30 aa, Bombyx mori cytoplasmic polyhedrosis virus
MADVAGTSNRDFRGREQRLFNSEQYNYNNS

SEQ ID NO: 5 - protein, 273 aa, synthetic construct
  REGION 1..273: note = synthesized protein
MADVAGTSNRDFRGREQRLFNSEQYNYNNSGSIASMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 6 - protein, 8 aa, Bombyx mori cytoplasmic polyhedrosis virus
EYREGQHN

SEQ ID NO: 7 - protein, 10 aa, synthetic construct
  REGION 1..10: note = synthesized sequence
YYDPETGTWY

SEQ ID NO: 8 - protein, 250 aa, synthetic construct
  REGION 1..250: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKALKYYDPETGTWYDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 9 - protein, 253 aa, synthetic construct
  REGION 1..253: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKALKEYYYDPETGTWYNDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 10 - protein, 256 aa, synthetic construct
  REGION 1..256: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKALKEYRYYDPETGTWYQHNDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 11 - protein, 18 aa, synthetic construct
  REGION 1..18: note = synthesized sequence
GGGSYYDPETGTWYGGGS

SEQ ID NO: 12 - protein, 258 aa, synthetic construct
  REGION 1..258: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKALKGGGSYYDPETGTWYGGGSDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 13 - protein, 4 aa, synthetic construct
  REGION 1..4: note = synthesized sequence
GGGS

SEQ ID NO: 14 - protein, 18 aa, synthetic construct
  REGION 1..18: note = synthesized sequence
EAAKYYDPETGTWYEAAK

SEQ ID NO: 15 - protein, 258 aa, synthetic construct
  REGION 1..258: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKALKEAAKYYDPETGTWYEAAKDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 16 - protein, 4 aa, synthetic construct
  REGION 1..4: note = synthesized sequence
EAAK

SEQ ID NO: 17 - protein, 245 aa, synthetic construct
  REGION 1..245: note = derived from Bombyx mori cytoplasmic polyhedrosis virus
MADVAGTSNRDFRGREQRLFNSEQYNYNNSLNGEVSVWVYAYYSDGSVLVINKNSQYKVGISETFKALKEYREGQHNDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVHNVMDVFQSCDLALRFCNRYWAELELVNHYISPNAYPYLDINNHSYGVALSNRQ

SEQ ID NO: 18 - protein, 644 aa, Bacillus thuringiensis
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 19 - protein, 340 aa, Trypanosoma brucei
MHLMRACITFCIASTAVVAVNAALVAEDAPVLSKAFVDRVNRLNRGIWKAKYDGVMQNITLREAKRLNGVIKKNNNASILPKRRFTEEEARAPLPSSFDSAEAWPNCPTIPQIADQSACGSCWAVAAASAMSDRFCTMGGVQDVHISAGDLLACCSDCGDGCNGGDPDRAWAYFSSTGLVSDYCQPYPFPHCSHHSKSKNGYPPCSQFNFDTPKCNYTCDDPTIPVVNYRSWTSYALQGEDDYMRELFFRGPFEVAFDVYEDFIAYNSGVYHHVSGQYLGGHAVRLVGWGTSNGVPYWKIANSWNTEWGMDGYFLIRRGSSECGIEDGGSAGIPLAPNTA

SEQ ID NO: 20 - protein, 548 aa, Luciola mingrelica
MEMEKEENVVYGPLPFYPIEEGSAGIQLHKYMHQYAKLGAIAFSNALTGVDISYQEYFDITCRLAEAMKNFGMKPEEHIALCSENCEEFFIPVLAGLYIGVAVAPTNEIYTLRELNHSLGIAQPTIVFSSRKGLPKVLEVQKTVTCIKKIVILDSKVNFGGHDCMETFIKKHVELGFQPSSFVPIDVKNRKQHVALLMNSSGSTGLPKGVRITHEGAVTRFSHAKDPIYGNQVSPGTAILTVVPFHHGFGMFTTLGYFACGYRVVMLTKFDEELFLRTLQDYKCTSVILVPTLFAILNKSELIDKFDLSNLTEIASGGAPLAKEVGEAVARRFNLPGVRQGYGLTETTSAFIITPEGDDKPGASGKVVPLFKVKVIDLDTKKTLGVNRRGEICVKGPSLMLGYSNNPEATRETIDEEGWLHTGDIGYYDEDEHFFIVDRLKSLIKYKGYQVPPAELESVLLQHPNIFDAGVAGVPDPDAGELPGAVVVMEKGKTMTEKEIVDYVNSQVVNHKRLRGGVRFVDEVPKGLTGKIDAKVIREILKKPQAKM

SEQ ID NO: 21 - protein, 311 aa, Renilla reniformis
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 22 - protein, 635 aa, Avian orthoreovirus
MASTKWGDKPMSLSMSHDGSSIRSAASQFLSVPLSHSTPIPPQRKTVLLKFMIGDDLVTVQGALAPFDEYWYDNQPLLAQAVEMLASEDRLRQFEHYEKFLLKKGHQIAEIMNRLRLFFTDVLKVKMEAEALPALAQYLMVGTLEAVSTAHSPDACVPVTSKVVTKQQTIAKSPGRLDEEEYNVIRSRFLTHEVFDLTSDLPGVQPFMDMYYATVPRADSTGWCVYRRKGLLIHSPDEQFSDLTIFSTRLTASHELQLVAGDVVVACFDLMDVSDIAPSHHASVQEERTLGTSKYSNITANDHPLVFFSPSALRWAIDHACTDSLVSTRNIRVCVGIDPLVTRWTRDGVQEAAILMDDKLPSAGRARMALRTLLLARRSPMPSFLLGALKQSGGQLLEHYRCDAANRYGSPTVPMSHPPPCSKCPELKEQITKLSSSPTPKIDSTTGPAALLSKISDLQRANRELSLKLVDMQPAREDHLLSYLNEHVCVNARDHEKGLLSRCNVSNESISSILDQRMKNRERFETRLRHEASAEWEPRVEALNQELAKARVEQQDMMTQSLQYLNERDELLHEVDELKRELTTLRAANVRLNADNHRMSRATRVGDAFVSDIEPLPSGIPGESKPSMEELVDDL

SEQ ID NO: 23 - protein, 387 aa, unidentified entomopoxvirus
MFIKILPILILFLDYVSGHGYITFPIARQRRCNVQGGFWWPPGGSGIPDPMCRAAYQNVYNKVLQQGGTIDQAASAAQYMFQQDNEYAALAGPNYLDQNHIRNNVVPNYLCAAHATTWRIRPFGDKTGMDVSGSWTPTVIPLQDNTVSTVPIEFEFCPTAIHEPSFFEIYITVPSFNVYTDQVTWQQLINIFTGPIPLVQRRPDSQCNAHNLVYRTTVGIPVRQTQFVLYVRWQRNDPVGEGFYNCADVIFAHRLGINEEDKIRPPKMKCKGNDKDCYKHHHRHNRYENDYENNYENYENYENNYENNYENNYENNYEYEYEYDRNNREHYHKCKHHSCMQHNYYERQYNTKDFNYVEWNDDYSDYIEIIQDNRDMCDSTTKCCYKK

SEQ ID NO: 24 - protein, 104 aa, Photorhabdus luminescens
MINDMHPSLIKDKDIVDDVMLRSCKIIAMKVMPDKVMQVMVTVLMHDGVCEEMLLKWNLLDNRGMAIYKVLMEALCAKKDVKISTVGKVGPLGCDYINCVEISM

(19) United States
(12) Patent Application Publication
(10) Pub. No.: US 2026/0167666 A1
(43) Pub. Date: Jun. 18, 2026

(54) PRODUCTION OF PROTEIN SOLID MATERIAL

(71) Applicant: Institute of Science Tokyo, Tokyo (JP)

(72) Inventors: Takafumi Ueno, Tokyo (JP); Satoshi Abe, Tokyo (JP); Mariko Kojima, Tokyo (JP)

(21) Appl. No.: 19/437,948

(22) Filed: Dec. 31, 2025

Related U.S. Application Data
(62) Division of application No. 17/632,739, now Pat. No. 12,540,159.

(30) Foreign Application Priority Data
Aug. 7, 2019 (JP) 2019-145456

(51) Int. Cl. C07K 1/30; C07K 1/02; C12N 15/62; C12P 21/02

(52) U.S. Cl. CPC C07K 1/306; C07K 1/02; C12N 15/62; C12P 21/02; C07K 2299/00

(57) ABSTRACT

A method of producing a protein crystal including adding, to a protein synthesis system, a nucleic acid that encodes crystalline protein; and incubating the protein synthesis system during the predetermined time until the crystalline protein encoded by the added nucleic acid is expressed and the expressed crystalline protein completes the formation of crystals. The protein synthesis system is a cell-free protein synthesis system.

### RELATED APPLICATIONS

The application is a divisional of U.S. patent application Ser. No. 17/632,739, filed Feb. 3, 2022, which is the U.S. national phase entry under 35 U.S.C. 371 of International Application No. PCT/JP2020/030226, filed Aug. 6, 2020, which claims priority to Japanese Patent Application No. 2019-145456, filed Aug. 7, 2019, the entire content of which is incorporated herein by reference.

### SEQUENCE LISTING STATEMENT

The present application contains a Sequence Listing, which is being submitted via EFS Web on even date herewith. The Sequence Listing is submitted in a file entitled “SHIGA029002D1SequenceListing.xml,” which was created on Dec. 28, 2025, and is approximately 32,506 bytes in size. This Sequence Listing is hereby incorporated by reference.

### TECHNICAL FIELD

The present invention relates to the production of a protein solid material.

### BACKGROUND OF THE INVENTION

Pathogens that cause polyhedrosis in insects such as silkworms include nuclear polyhedrosis virus (Nucleopolyhedrovirus, NPV), which is a pathogenic virus for nuclear polyhedrosis, and cytoplasmic polyhedrosis virus (Cypovirus, CPV), which is a pathogenic virus for cytoplasmic polyhedrosis. The former is a DNA virus, while the latter is an RNA virus. NPV has been widely utilized by many researchers as a baculovirus vector.

The polyhedrosis viruses produce, in the late stage of infection, inclusion bodies called polyhedra in infected cells in a large quantity that reaches about half the total quantity of cellular proteins, and enclose a large number of virus particles in the inclusion bodies. The viruses enclosed in the polyhedra are protected from the inactivating action from the external environment, such as ultraviolet radiation and heat, and can maintain infectivity for an extended period of time.

The polyhedra are stable without being dissolved by many solvents and surfactants; however, the polyhedra are dissolved under alkaline conditions with a pH of about 10 or higher. When the virus particles enclosed in the polyhedra are eaten by insects, the polyhedra are dissolved by the high pH of the intestine, and the virus particles are released, causing infection.

A polyhedron as mentioned above is a product obtained by spontaneous crystallization of polyhedrin, which is a polyhedral protein, in a cell. In addition to polyhedrin, for example, an insecticidal protein of Bacillus thuringiensis and the like are known as proteins that are spontaneously crystallized in cells.

In the present specification, a protein that spontaneously forms a crystal is referred to as a crystalline protein, and a crystal formed as the result is referred to as a protein crystal.

Incidentally, in recent years, studies for utilizing polyhedra have been carried out. For example, in Patent Document 1, a method for producing a polyhedron-target molecule complex in which a target molecule is enclosed in a polyhedron is described. Furthermore, in Non Patent Document 1, it is described that when a gene encoding a fusion protein with the H1α-helix present at the N-terminus of the polyhedrin protein, which is a polyhedral protein, is co-expressed together with a gene encoding the polyhedrin protein in a cell, a polyhedron in which the fusion protein is enclosed in the polyhedron crystal of cytoplasmic polyhedrosis virus can be prepared.

### DOCUMENTS OF RELATED ART

### Patent Documents

Patent Document 1: Japanese Unexamined Patent Application, First Publication No. 2018-033404

### Non Patent Documents

Non Patent Document 1: Ijiri H., et al., Structure-based targeting of bioactive proteins into cypovirus polyhedra and application to immobilized cytokines for mammalian cell culture., Biomaterials, 30 (26), 4297-4308, 2009

### SUMMARY OF THE INVENTION

### Problems to be Solved by the Invention

However, the methods described in Patent Document 1 and Non Patent Document 1 require a great deal of time and labor in order to obtain crystals of a polyhedral protein. Therefore, the present invention provides a technology for obtaining protein crystals in a short period of time with little effort.

### Means to Solve the Problems

The present invention includes the following embodiments.
[1] A method for producing a protein crystal, the method including:
step (a) of adding a nucleic acid encoding a crystalline protein to a protein synthesis system; and
step (b) of incubating the protein synthesis system for a predetermined time until the crystalline protein encoded by the added nucleic acid is expressed and the expressed crystalline protein completes the formation of crystals,
in which the protein synthesis system is a cell-free protein synthesis system.
[2] The method for producing a protein crystal according to [1], in which the crystalline protein is a protein described in any one of the following (A) to (C):
(A) a cytoplasmic polyhedral protein, a nuclear polyhedral protein, an insecticidal protein, cathepsin B, an insecticidal protein, reovirus nonstructural protein (μNS), Crystalline Inclusion Protein A (CipA), or fusolin protein (Fusolin);
(B) a protein including an amino acid sequence in which one amino acid or a plurality of amino acids have been deleted, substituted, or added in an amino acid sequence of the protein of (A), the protein having a crystal-forming ability; and
(C) a fusion protein of the protein of (A) or (B) and a target peptide.
[3] The method for producing a protein crystal according to [1], in which the crystalline protein is one selected from the group consisting of the following (i), (ii), (iii) and (iv):
(i) a protein including an amino acid sequence set forth in SEQ ID NO:1, or a protein including an amino acid sequence in which one amino acid or a plurality of amino acids have been deleted, substituted, or added in the amino acid sequence set forth in SEQ ID NO: 1, the protein having a polyhedron-forming ability;
(ii) a protein including an amino acid sequence set forth in SEQ ID NO:2, or a protein including an amino acid sequence in which one amino acid or a plurality of amino acids have been deleted, substituted, or added in the amino acid sequence set forth in SEQ ID NO:2, the protein having a polyhedron-forming ability;
(iii) a protein including an amino acid sequence set forth in SEQ ID NO:18, or a protein including an amino acid sequence in which one amino acid or a plurality of amino acids have been deleted, substituted, or added in the amino acid sequence set forth in SEQ ID NO: 18, the protein having a crystal-forming ability; and
(iv) a fusion protein in which an amino acid sequence of the 70th amino acid to the 77th amino acid of the amino acid sequence set forth in SEQ ID NO:1 has been substituted with a target peptide, the fusion protein having a polyhedron-forming ability, or a protein including an amino acid sequence in which one amino acid or a plurality of amino acids have been deleted, substituted, or added in the fusion protein, the protein having a polyhedron-forming ability.
[4] The method for producing a protein crystal according to [3], in which the crystalline protein is (i) or (iv), and in the step (a), a nucleic acid encoding a fusion protein in which a target peptide is bonded to the C-terminus of an amino acid sequence set forth in SEQ ID NO:4 or to the C-terminus of an amino acid sequence in which one amino acid or a plurality of amino acids have been deleted, substituted, or added in the amino acid sequence set forth in SEQ ID NO:4, is further added to the protein synthesis system.
[5] The method for producing a protein crystal according to any one of [1] to [4], in which the cell-free protein synthesis system includes a target molecule having a molecular weight of 10 to 100,000.
[6] A fusion protein having an amino acid sequence of the 70th amino acid to the 77th amino acid of the amino acid sequence set forth in SEQ ID NO:1 substituted with a target peptide, the fusion protein having a polyhedron-forming ability, or a fusion protein including an amino acid sequence in which one amino acid or a plurality of amino acids have been deleted, substituted, or added in the fusion protein, the fusion protein having a polyhedron-forming ability.
[7] A polyhedron, in which the fusion protein according to [6] has been crystallized.
[8] A polyhedron-target molecule complex, in which a target molecule is enclosed in a crystal structure of the crystallized polyhedron according to [7].
[9] A nucleic acid encoding the fusion protein according to [6].
[10] A kit for producing a cell-free system crystal, the kit including: a nucleic acid encoding a crystalline protein; and a reagent for a cell-free protein synthesis system.
[11] The kit for producing a cell-free system crystal according to [10], in which the crystalline protein is a protein described in any one of the following (A) to (C):
(A) a cytoplasmic polyhedral protein, a nuclear polyhedral protein, an insecticidal protein, cathepsin B, an insecticidal protein, luciferase, reovirus nonstructural protein (uNS), Crystalline Inclusion Protein A (CipA), or fusolin protein (Fusolin);
(B) a protein including an amino acid sequence in which one amino acid or a plurality of amino acids have been deleted, substituted, or added in an amino acid sequence of the protein of (A), the protein having a crystal-forming ability; and
(C) a fusion protein of the protein of (A) or (B) and a target peptide.
[12] The kit for producing a cell-free system crystal according to [10], in which the crystalline protein is one selected from the group consisting of the following (i), (ii), (iii) and (iv):
(i) a protein including an amino acid sequence set forth in SEQ ID NO:1, or a protein including an amino acid sequence in which one amino acid or a plurality of amino acids have been deleted, substituted, or added in the amino acid sequence set forth in SEQ ID NO:1, the protein having a polyhedron-forming ability;
(ii) a protein including an amino acid sequence set forth in SEQ ID NO:2, or a protein including an amino acid sequence in which one amino acid or a plurality of amino acids have been deleted, substituted, or added in the amino acid sequence set forth in SEQ ID NO: 1, the protein having a polyhedron-forming ability;
(iii) a protein including an amino acid sequence set forth in SEQ ID NO:18, or a protein including an amino acid sequence in which one amino acid or a plurality of amino acids have been deleted, substituted, or added in the amino acid sequence set forth in SEQ ID NO: 18, the protein having a crystal-forming ability; and
(iv) a fusion protein in which an amino acid sequence of the 70th amino acid to the 77th amino acid of the amino acid sequence set forth in SEQ ID NO: 1 has been substituted with a target peptide, the fusion protein having a polyhedron-forming ability, or a protein including an amino acid sequence in which one amino acid or a plurality of amino acids have been deleted, substituted, or added in the fusion protein, the protein having a polyhedron-forming ability.
[13] The kit for producing a cell-free system crystal according to [12], in which the crystalline protein is (i) or (iv), and
the kit further includes a nucleic acid encoding a fusion protein in which a target peptide is bonded to the C-terminus of an amino acid sequence set forth in SEQ ID NO: 4 or to the C-terminus of an amino acid sequence in which one amino acid or a plurality of amino acids have been deleted, substituted, or added in the amino acid sequence set forth in SEQ ID NO:4.

### Effects of the Invention

According to the present invention, a technology for obtaining protein crystals in a short period of time with less efforts can be provided.

### BRIEF DESCRIPTION OF DRAWINGS

FIG. 1 is an optical microscopic image of polyhedra crystals obtained by a cell-free protein synthesis system.

FIG. 2 is SEM images of polyhedra crystals obtained by a cell-free protein synthesis system at 15° C.

FIG. 3 is SEM images of polyhedra crystals obtained by a cell-free protein synthesis system at 20° C.

FIG. 4 shows results obtained by analyzing a three-dimensional structure of a polyhedra crystals obtained by a cell-free protein synthesis system, by an X-ray structural analysis.

FIG. 5A is SEM images of crystals obtained from 220 μL of a solution and 55 μL of the solution.

FIG. 5B is optical microscopic images of crystals obtained from 22 μL of the solution.

FIG. 5C is SEM images of crystals obtained from 22 μL of the solution.

FIG. 6A is a fluorescence image of GFP captured by irradiating polyhedra crystals enclosing GFP, with excitation light.

FIG. 6B is a result obtained by superimposing a bright field image and a fluorescence image of the crystals.

FIG. 7 is a fluorescence image captured by irradiating polyhedra crystals enclosing fluorescein, with excitation light.

FIG. 8A is a schematic diagram of a three-dimensional structure of polyhedrin protein in the vicinity of a substituted peptide.

FIG. 8B is a schematic diagram of the three-dimensional structure of polyhedrin protein in the vicinity of the substituted peptide.

FIG. 8C is a schematic diagram of the three-dimensional structure of polyhedrin protein in the vicinity of the substituted peptide.

FIG. 8D is a schematic diagram of the three-dimensional structure of polyhedrin protein in the vicinity of the substituted peptide.

FIG. 9 is images captured by optical microscopy and images taken by SEM of a crystal formed from a mutant polyhedrin protein.

FIG. 10 is a histogram of the sizes of crystals formed from mutant polyhedrin proteins.

FIG. 11A is a photograph of a capillary tube used.

FIG. 11B shows crystals of a mutant polyhedrin protein formed in the capillary tube.

FIG. 12 shows results obtained by mass spectrometry of crystals formed from mutant polyhedrin proteins.

FIG. 13 shows results obtained by solubilizing crystals formed from mutant polyhedrin proteins and analyzing them by SDS-PAGE.

FIG. 14A is optical microscopic images of Cry3A protein crystals produced at 20° C.

FIG. 14B is optical microscopic images of Cry3A protein crystals produced at 4° C.

FIG. 14C is SEM images of Cry3A protein crystals produced at 20° C.

FIG. 14D is SEM images of Cry3A protein crystals produced at 4° C.

FIG. 15A is optical microscopic images of μNS protein crystals produced at 20° C.

FIG. 15B is optical microscopic images of μNS protein crystals produced at 4° C.

FIG. 15C is SEM images of μNS protein crystals produced at 20° C.

FIG. 15D is SEM images of μNS protein crystals produced at 4° C.

FIG. 16A is optical microscopic images of cathepsin B protein crystals produced at 20° C.

FIG. 16B is optical microscopic images of cathepsin B protein crystals produced at 4° C.

FIG. 16C is SEM images of cathepsin B protein crystals produced at 20° C.

FIG. 16D is SEM images of cathepsin B protein crystals produced at 4° C.

FIG. 17A is optical microscopic images of nuclear polyhedral protein crystals produced at 20° C.

FIG. 17B is optical microscopic images of nuclear polyhedral protein crystals produced at 4° C.

FIG. 17C is SEM images of nuclear polyhedral protein crystals produced at 20° C.

FIG. 17D is SEM images of nuclear polyhedral protein crystals produced at 4° C.

FIG. 18 is optical microscopic images of CipA protein crystals produced at 20° C.

### EMBODIMENTS FOR CARRYING OUT THE INVENTION

### Production Method

According to an embodiment, the present invention provides a method for producing a protein crystal, the method including: step (a) of adding a nucleic acid encoding a crystalline protein to a protein synthesis system; and step (b) of incubating the protein synthesis system for a predetermined time until the crystalline protein encoded by the added nucleic acid is expressed and the expressed crystalline protein completes the formation of crystals, in which the protein synthesis system is a cell-free protein synthesis system.

According to the present specification, the crystalline protein means a protein capable of spontaneous self-assembling to form crystals under physiological conditions such as in the cytoplasm. Furthermore, according to the present specification, the protein crystal means a crystal obtained by crystallizing the above-mentioned crystalline protein.

The crystalline protein is not particularly limited as long as it is a protein that forms a crystal in a cell-free protein synthesis system. Even for a non-crystalline protein that originally does not form a crystal, when the non-crystalline protein becomes capable of forming a crystal in a cell-free protein synthesis system by means of techniques such as chemical modification of the protein, creation of a variant, and a fusion protein, this protein is also included in the crystalline protein according to the present specification.

More limited examples of the crystalline protein include the proteins described in any of the following (A) to (C):
(A) a cytoplasmic polyhedral protein, a nuclear polyhedral protein, an insecticidal protein, cathepsin B, luciferase, reovirus nonstructural protein (μNS), Crystalline Inclusion Protein A (CipA), or fusolin protein (Fusolin);
(B) a protein including an amino acid sequence in which one amino acid or a plurality of amino acids have been deleted, substituted, or added in an amino acid sequence of the protein of (A), the protein having a crystal-forming ability; and
(C) a fusion protein of the protein of (A) or (B) and a target peptide.

The cytoplasmic polyhedral protein is a polyhedrin protein derived from a cytoplasmic polyhedrosis virus (Cypovirus, CPV), which is a pathogenic virus for cytoplasmic polyhedrosis. For example, a protein including an amino acid sequence set forth in SEQ ID NO: 1 is wild-type polyhedrin protein expressed by a cytoplasmic polyhedrosis virus that infects silkworm moth.

The nuclear polyhedral protein is polyhedrin protein derived from a nuclear polyhedrosis virus (Nucleopolyhedrovirus, NPV), which is a pathogenic virus for nuclear polyhedrosis. For example, a protein including an amino acid sequence set forth in SEQ ID NO: 2 is wild-type polyhedrin protein expressed by a nuclear polyhedrosis virus that infects Autographa californica.

The insecticidal protein is an insecticidal protein produced by Bacillus thuringiensis. An amino acid sequence of Cry3A protein produced by Bacillus thuringiensis is set forth in SEQ ID NO: 18.

Cathepsin B is a protease having endopeptidase activity and exopeptidase activity. Cathepsin B is a protein that forms a crystal in cultured insect cells. An amino acid sequence of Trypanosoma brucei-derived cathepsin B is set forth in SEQ ID NO:19.

Luciferase is a generic name for enzymes that have the action of catalyzing a chemical reaction in which a luminescent material emits light in bioluminescence of luminescent bacteria, fireflies, and the like. Luciferase is a protein that forms a crystal in insect cells. An amino acid sequence of firefly-derived luciferase is set forth in SEQ ID NO:20, and an amino acid sequence of sea pansy-derived luciferase is set forth in SEQ ID NO:21.

μNS is reovirus nonstructural protein having crystallinity. An amino acid sequence of reovirus-derived μNS is set forth in SEQ ID NO:22.

Fusolin is a constituent protein of a crystalline protein inclusion body formed in host cells by Entomopoxvirus. An amino acid sequence of Entomopoxvirus-derived Fusolin is set forth in SEQ ID NO:23.

Crystalline inclusion protein A (CipA) is a constituent protein of a crystalline protein inclusion body of the genus Photorhabdus. An amino acid sequence of Photorhabdus laumondii-derived CipA is set forth in SEQ ID NO:24.

According to the present specification, the phrase one or a plurality means, for example, 1 to 50 units, for example, 1 to 35 units, for example, 1 to 20 units, for example, 1 to 15 units, for example, 1 to 10 units, and for example, 1 to 5 units.

The crystalline protein may be a variant having a mutation in the above-mentioned cytoplasmic polyhedral protein, nuclear polyhedral protein, insecticidal protein, cathepsin B, luciferase, μNS, CipA, Fusolin, and the like as long as it has a crystal-forming ability. More specifically, the crystalline protein may be, for example, a protein including an amino acid sequence in which one amino acid or a plurality of amino acids have been deleted, substituted, or added in the above-mentioned amino acid sequences set forth in SEQ ID NO:1, SEQ ID NO:2, and SEQ ID NO: 18 to SEQ ID NO:24.

Here, the target peptide may be, for example, a peptide whose three-dimensional structure is to be analyzed. As will be described later, the three-dimensional structure of a target peptide can be conveniently analyzed by causing a crystalline protein, which is a fusion protein of the above-mentioned crystalline protein and a target peptide, to be expressed in a cell-free protein synthesis system to form a crystal, and subjecting the crystal of the crystalline protein to an X-ray crystal structure analysis.

In this case, the target peptide may be any peptide in need of analysis of the three-dimensional structure. The amino acid length of the target peptide is preferably, for example, about 5 to 50 amino acids from the viewpoint that the crystalline protein can maintain the crystal-forming ability.

Regarding a method for crystallizing a protein, a method of purifying the protein from cells and then performing crystallization is generally used. According to such a method, a duration of two weeks or longer was required in order to obtain crystals, and a lot of effort was also required.

For example, in order to produce polyhedra crystals, a method of infecting cells with a baculovirus to produce polyhedra crystals in the cells may be adopted. In this case, a duration of about 2 weeks was required to prepare the baculovirus.

On the other hand, in the case of the method for producing a crystal using a cell-free protein synthesis system of the present embodiment, the time required for expressing and crystallizing a crystalline protein is about one day. Furthermore, according to the method for producing a protein crystal of the present embodiment, the operation is very simple and requires little effort.

Furthermore, in the cell-free protein synthesis system, the amount of the synthesis system can be freely set. As will be described later in the Examples, for example, protein crystals can be obtained even when the amount of the reaction solution of the cell-free protein synthesis system is about 22 μL.

The lower limit value of the amount of the reaction solution of the cell-free protein synthesis system is preferably 15 UL or more, more preferably 18 μL or more, even more preferably 20 μL or more, and particularly preferably 22 μL or more. When the amount of the reaction solution is equal to or more than the above-described lower limit value, the shape of the protein crystals can be made more well-regulated.

The upper limit value of the amount of the reaction solution of the cell-free protein synthesis system is not particularly limited, and the amount of the reaction solution can be a liquid amount capable of forming protein crystals. The amount of the reaction solution may be, for example, 5 mL or less, 1 mL or less, 500 μL or less, 200 μL or less, 100 μL or less, 50 μL or less, or 30 μL or less.

When a crystalline protein is synthesized in a cell, it is necessary to synthesize the crystalline protein at a temperature and an environment suitable for the proliferation and growth of the cell. On the other hand, when a crystalline protein is synthesized in a cell-free protein synthesis system, the temperature, environment, and the like can be appropriately set. For example, the quality of the obtained crystals can be enhanced by synthesizing the crystalline protein using a cell-free protein synthesis system at a temperature lower than the general culture temperature for the cells.

Furthermore, as will be described later in the Examples, protein crystals can be obtained, even when the amount of the reaction solution is 20 μL or less, by injecting the reaction solution of the cell-free protein synthesis system into a capillary tube. When protein crystals are obtained using a capillary tube, the amount of the reaction solution to be injected into the capillary tube is preferably 5 μL or more, more preferably 12 μL or more, even more preferably 15 μL or more, and particularly preferably 18 μL or more. When the amount of the reaction solution is equal to or more than the above-described lower limit value, the shape of the protein crystals can be made more well-regulated.

The upper limit value of the amount of the reaction solution of the cell-free protein synthesis system to be injected into the capillary tube is not particularly limited, and the amount of the reaction solution can be a liquid amount which can form protein crystals and can be retained in the capillary tube. The amount of the reaction solution may be, for example, 100 μL or less, 50 μL or less, or 20 μL or less.

In the production method of the present embodiment, a nucleic acid encoding the crystalline protein is added to the protein synthesis system in step (a).

### (Nucleic Acid)

According to the present embodiment, the nucleic acid encoding the crystalline protein may be DNA or may be RNA. The base sequence of the nucleic acid encoding the protein may be synonymously substituted such that the expression of the crystalline protein is optimized in the cell-free protein synthesis system that will be described later.

According to the present embodiment, the crystalline protein to be expressed in the same cell-free protein synthesis system may be of one kind or may be of two or more kinds. According to the present embodiment, the nucleic acid to be added to the cell-free protein synthesis system may be of one kind or may be of two or more kinds.

The sequence of DNA may have a promoter for the binding of RNA polymerase upstream of the sequence encoding the crystalline protein. The promoter may be a promoter having activity in the protein synthesis system or may be an expression-inducing promoter whose activity can be induced by a drug or the like. The promoter is not particularly limited, and examples thereof include T3, T7, and SP6 promoters, cytomegalovirus promoter (CMV promoter), and CMV early enhancer/chicken beta actin (CAG promoter). Examples of the expression-inducing promoter include a doxycycline-induced promoter (TetO promoter), whose promoter activity can be artificially controlled.

The above-mentioned RNA may have a ribosome-binding site upstream of the sequence encoding the crystalline protein. Examples of the ribosome-binding site include Shine-Dalgarno sequence, Kozak sequence, and internal ribosome entry site (IRES); however, sequences known to those ordinarily skilled in the art can be used without limitations.

In step (b) of the present embodiment, the protein synthesis system is incubated for a predetermined time until the crystalline protein encoded by the added nucleic acid is expressed and the expressed protein completes the formation of crystals. Here, the predetermined time may be 10 minutes or more, 30 minutes or more, 1 hour or more, 3 hours or more, 6 hours or more, 12 hours or more, 24 hours or more, 32 hours or more, or 48 hours or more. The predetermined time may be 64 hours or less, 48 hours or less, 32 hours or less, 24 hours or less, 12 hours or less, 6 hours or less, 3 hours or less, 1 hour or less, or 30 minutes or less. The upper limit value and the lower limit value of the predetermined time can be discretionarily selected.

In step (b) of the present embodiment, the temperature of the protein synthesis system may be, for example, 3° C. or higher, 4° C. or higher, 10° C. or higher, 15° C. or higher, or 20° C. or higher. Furthermore, the temperature may be, for example, 35° C. or lower, 30° C. or lower, 25° C. or lower, 20° C. or lower, 15° C. or lower, 10° C. or lower, or 5° C. or lower. The upper limit value and the lower limit value of the temperature can be discretionarily selected.

When the reaction solution of the cell-free protein synthesis system is incubated for a predetermined time, protein crystals are formed on the bottom of the container holding the reaction solution. The formed crystals can be easily collected by an operation such as centrifugation.

### (Cell-Free Protein Synthesis System)

According to the present embodiment, the protein synthesis system is a cell-free protein synthesis system. It is meant by a cell-free protein synthesis system that proteins are not intracellularly synthesized but are synthesized in vitro from nucleic acids that serve as templates, by using ribosomes, transcriptional/translation factors, and the like, which are derived from living cells or artificially synthesized.

The cell-free protein synthesis system of the present embodiment may be a synthesis system including a transcription process in addition to the translation process. When the nucleic acid encoding the protein having a crystal-forming ability of the present embodiment is DNA, it is necessary to synthesize RNA encoding the protein having a crystal-forming ability, by transcribing the DNA.

A step of transcribing DNA may be included in step (b) of the present embodiment. That is, the cell-free protein synthesis system of the present embodiment may include a factors that enable transcription. Examples of the factors that enable transcription include RNA polymerases and nucleotides; however, factors known to those ordinarily skilled in the art can be used without limitations.

Furthermore, the above-mentioned process of transcribing RNA from DNA does not have to be included in the process of the present embodiment. That is, RNA may be synthesized in advance using a DNA encoding a crystalline protein as a template, and the RNA may be added to the cell-free protein synthesis system of the present embodiment. Furthermore, instead of using the DNA encoding the crystalline protein, it is also acceptable to use an artificially chemically synthesized RNA encoding the crystalline protein.

The cell-free protein synthesis system is not particularly limited, and examples thereof include a synthesis system that utilizes a cell extract obtained from wheat germ, yeast, insect cells, cultured mammalian cells, rabbit reticulocytes, Escherichia coli, or the like; and a synthesis system in which factors required for translation have been reconstituted. The cell-free protein synthesis system used in the present embodiment is not limited to the above-mentioned methods, and known methods can be used (see, for example, Japanese Unexamined Patent Application, First Publication No. 2019-083825).

A cell extract used in the cell-free protein synthesis system may include, for example, tRNA, aminoacylation tRNA synthetase, a translation initiation factor, a translation elongation factor, and a translation termination factor, as factors involved in translation.

The efficiency of protein synthesis may be increased by continuously adding, for example, amino acids and energy molecules (ATP, GTP) to the cell extract. Furthermore, for example, an energy regeneration system, salts, creatine phosphate, and an enzyme may be added to the cell extract as needed.

The synthesis system in which factors required for translation have been reconstituted is not particularly limited, and for example, a known method can be used. (For example, Shimizu, Y. et al., Cell-free translation reconstituted with purified components, Nature Biotech., 19, 751-755, 2001). The reconstitution type synthesis system may include, for example, a translation initiation factor, a translation elongation factor, a termination factor, aminoacylation tRNA synthetase, and methionyl-tRNA formyltransferase.

For example, the following operations may be carried out using the cell-free protein synthesis system. As described above, factors that regulate translation may be added to the cell-free protein synthesis system as necessary. Furthermore, the pH, salt concentration, and the like of the synthesis system can be changed as necessary.

Furthermore, as will be described later, a target substance can be enclosed in the polyhedra by adding the target substance to the cell-free protein synthesis system.

Furthermore, by adding, for example, an unnatural amino acid, a radiolabeled amino acid, and the like to the cell-free protein synthesis system, the unnatural amino acid and the radiolabeled amino acid can be incorporated into the polyhedrin protein of the polyhedra.

In addition, compounds harmful and toxic to cells can also be added to the cell-free protein synthesis system.

Furthermore, when the cell-free protein synthesis system is used, a large quantity of crystalline proteins can be synthesized. As described above, for example, by adding a factor that enhances the efficiency of protein synthesis to the cell-free protein synthesis system, a large quantity of crystalline proteins can be synthesized.

The crystalline protein described above may be one selected from the group consisting of the following (i), (ii), (iii), and (iv):
(i) a protein including an amino acid sequence set forth in SEQ ID NO:1, or a protein including an amino acid sequence in which one amino acid or a plurality of amino acids have been deleted, substituted, or added in the amino acid sequence set forth in SEQ ID NO:1, the protein having a polyhedron-forming ability;
(ii) a protein including an amino acid sequence set forth in SEQ ID NO:2, or a protein including an amino acid sequence in which one amino acid or a plurality of amino acids have been deleted, substituted, or added in the amino acid sequence set forth in SEQ ID NO:2, the protein having a crystal-forming ability;
(iii) a protein including an amino acid sequence set forth in SEQ ID NO:18, or a protein including an amino acid sequence in which one amino acid or a plurality of amino acids have been deleted, substituted, or added in the amino acid sequence set forth in SEQ ID NO: 18, the protein having a crystal-forming ability; and
(iv) a fusion protein in which an amino acid sequence of the 70th amino acid to the 77th amino acid of the amino acid sequence set forth in SEQ ID NO: 1 has been substituted with a target peptide, the fusion protein having a polyhedron-forming ability, or a protein including an amino acid sequence in which one amino acid or a plurality of amino acids have been deleted, substituted, or added in the fusion protein, the protein having a polyhedron-forming ability.

These will be described in detail.

According to the present specification, the phrase one or a plurality means, for example, 1 to 50 units, for example, 1 to 35 units, for example, 1 to 20 units, for example, 1 to 15 units, for example, 1 to 10 units, and for example, 1 to 5 units.

### First Embodiment

The crystalline protein may be the following (i).
(i) a protein including an amino acid sequence set forth in SEQ ID NO:1, or a protein including an amino acid sequence in which one amino acid or a plurality of amino acids have been deleted, substituted, or added in the amino acid sequence set forth in SEQ ID NO: 1, the protein having a polyhedron-forming ability;

The protein including the amino acid sequence set forth in SEQ ID NO: 1 is wild-type polyhedrin protein expressed by Cytoplasmic Polyhedrosis Virus (CPV) that infects silkworm moth. Wild-type polyhedrin protein has a polyhedron-forming ability of forming polyhedra by spontaneously self-assembling.

As will be described later in Examples, a polyhedra protein crystal can be produced by causing the crystalline protein of the above-described item (i) to be expressed in the above-mentioned cell-free protein synthesis system and incubating the cell-free protein synthesis system.

### Second Embodiment

The crystalline protein may be the following (ii).
(ii) a protein including an amino acid sequence set forth in SEQ ID NO:2, or a protein including an amino acid sequence in which one amino acid or a plurality of amino acids have been deleted, substituted, or added in the amino acid sequence set forth in SEQ ID NO: 2, the protein having a polyhedron-forming ability;

The protein including the amino acid sequence set forth in SEQ ID NO:2 is wild-type polyhedrin protein expressed by nuclear polyhedrosis virus (Nucleopolyhedrovirus, NPV) that infects Autographa californica, which is a type of moth. This wild-type polyhedrin protein has a crystal-forming ability to form protein crystals by spontaneous self-assembling.

A polyhedra protein crystal can be produced by causing the crystalline protein of the above-described item (ii) to be expressed in the above-mentioned cell-free protein synthesis system and incubating the cell-free protein synthesis system.

### Third Embodiment

The crystalline protein may be the following (iii).
(iii) a protein including an amino acid sequence set forth in SEQ ID NO:18, or a protein including an amino acid sequence in which one amino acid or a plurality of amino acids have been deleted, substituted, or added in the amino acid sequence set forth in SEQ ID NO: 18, the protein having a crystal-forming ability; and

The protein including the amino acid sequence set forth in SEQ ID NO: 18 is Cry3A protein produced by Bacillus thuringiensis. Wild-type Cry3A protein has a crystal-forming ability of forming protein crystals by spontaneously self-assembling.

As will be described later in Examples, crystals of the crystalline protein can be produced by causing the crystalline protein of the above-described item (iii) to be expressed in the above-mentioned cell-free protein synthesis system and incubating the cell-free protein synthesis system.

### Fourth Embodiment

The crystalline protein may be the following (iv).
(iv) a fusion protein in which an amino acid sequence of the 70th amino acid to the 77th amino acid of the amino acid sequence set forth in SEQ ID NO:1 has been substituted with a target peptide, the fusion protein having a polyhedron-forming ability, or a protein including an amino acid sequence in which one amino acid or a plurality of amino acids have been deleted, substituted, or added in the fusion protein, the protein having a polyhedron-forming ability.

A crystal structure analysis of the target peptide can be easily performed by analyzing the crystal structure of polyhedra obtained by crystallizing the fusion protein of the present embodiment.

Furthermore, the size of the polyhedra crystals can be controlled by appropriately designing the amino acid sequence of the target peptide.

The number of amino acids in the amino acid sequence of the target peptide may be, for example, 5 to 25 residues, may be 7 to 23 residues, or may be 9 to 18 residues.

Examples of the fusion protein of the present embodiment include fusion proteins including amino acid sequences set forth in SEQ ID NO:8 to SEQ ID NO: 10, SEQ ID NO: 12, and SEQ ID NO: 15, as will be described later in Examples. The amino acid sequences of these fusion proteins will be described in detail.

The fusion protein of SEQ ID NO:8 is a fusion protein in which an amino acid sequence of the 70th amino acid to the 77th amino acid of the amino acid sequence set forth in SEQ ID NO:1 has been substituted with a target peptide including an amino acid sequence set forth in SEQ ID NO:7.

The fusion protein of SEQ ID NO:9 is a fusion protein in which two amino acid residues have been added on the N-terminal side of the target peptide and one amino acid residue has been added on the C-terminal side of the target peptide in the fusion protein of SEQ ID NO:8.

The fusion protein of SEQ ID NO: 10 is a fusion protein in which three amino acid residues have been added on the N-terminal side of the target peptide and three amino acid residues have been added on the C-terminal side of the target peptide in the fusion protein of SEQ ID NO:8.

The fusion protein of SEQ ID NO: 12 is a fusion protein in which four amino acid residues have been added on the N-terminal side of the target peptide and four amino acid residues have been added on the C-terminal side of the target peptide in the fusion protein of SEQ ID NO:8.

The fusion protein of SEQ ID NO: 15 is a fusion protein in which four amino acid residues have been added on the N-terminal side of the target peptide and four amino acid residues have been added on the C-terminal side of the target peptide in the fusion protein of SEQ ID NO:8.

As will be described later in Examples, a polyhedra crystals formed from the protein of SEQ ID NO: 12 has a size of about 400 nm, which is smaller compared with the size of a crystal formed from wild-type polyhedrin protein.

### Fifth Embodiment

According to an embodiment, the present invention provides a method for producing a protein crystal, in which the crystalline protein is the above-described item (i) or the above-described item (iv), and in step (a), a nucleic acid encoding a fusion protein in which a target peptide is bound to the C-terminus of an amino acid sequence set forth in SEQ ID NO: 4 or to the C-terminus of an amino acid sequence in which one amino acid or a plurality of amino acids have been deleted, substituted, or added in the amino acid sequence set forth in SEQ ID NO:, is further added to a cell-free protein synthesis system.

The amino acid sequence set forth in SEQ ID NO:4 is an H1α-helix region of wild-type cytoplasmic polyhedral protein.

According to the present embodiment, when a fusion protein and a protein having a polyhedron-forming ability are expressed by a cell-free protein synthesis system, these proteins form a polyhedron, and the fusion protein can form a portion of the polyhedron.

When the target peptide bound to the C-terminus of the fusion protein binds and the protein having a polyhedron-forming ability forms a polyhedron, the target peptide is enclosed in the polyhedron. As will be described later in Examples, when GFP is used as the target peptide is GFP, the polyhedron crystal can enclose GFP.

The sequence of the target peptide is not particularly limited. The number of residues in the amino acid sequence of the target peptide is preferably 3 to 500 residues, more preferably 3 to 400 residues, and even more preferably 3 to 300 residues.

### Sixth Embodiment

The crystalline protein may be a modified polyhedrin protein in which at least a portion of the amino acid sequence has been deleted from the protein of the following (v) or (vi), the modified polyhedrin protein having a polyhedon-forming ability.
(v) Wild-type polyhedrin protein
(vi) A protein including an amino acid sequence in which one amino acid or a plurality of amino acids have been deleted, substituted, or added in the amino acid sequence of wild-type polyhedrin protein, the protein having a polyhedron-forming ability.

The wild-type polyhedrin protein may be a wild-type polyhedrin protein expressed by CPV or a wild-type polyhedrin protein expressed by NPV.

More specifically, the wild-type polyhedrin protein may be, for example, a protein including an amino acid sequence set forth in SEQ ID NO:1 or a protein including an amino acid sequence set forth in SEQ ID NO:2.

A polyhedron obtained by crystallizing the modified polyhedrin protein of the present embodiment has a space inside. Therefore, it is easy to enclose a larger molecule, for which it has been hitherto difficult to be enclosed. According to the present specification, it should be noted that peptides include proteins.

According to the present embodiment, the modified polyhedrin protein may be a protein in which at least a portion of the amino acid sequence has been deleted from the wild-type polyhedrin protein.

Furthermore, according to the present embodiment, the modified polyhedrin protein may be a protein in which at least a portion of the amino acid sequence has been deleted from a protein including an amino acid sequence in which one amino acid or a plurality of amino acids have been deleted, substituted, or added in the amino acid sequence of the wild-type polyhedrin protein, the protein having a polyhedron-forming ability.

The total length of the at least portion of the amino acid sequence thus deleted may be 30 amino acids or more, may be 35 amino acids or more, or may be 40 amino acids or more. When more amino acids can be deleted while maintaining the polyhedron-forming ability, the space inside a polyhedron can be made larger.

The upper limit of the total length of the at least portion of the amino acid sequence thus deleted may be a length of the extent that the modified polyhedrin protein can maintain the polyhedron-forming ability, and the upper limit may be 100 amino acids for example, may be 80 amino acids for example, or may be 50 amino acids for example.

Furthermore, the deleted amino acid sequence may be at one location or may be at two or more locations, as long as the polyhedron-forming ability is maintained. That is, in the amino acid sequence of the wild-type polyhedrin protein that is the basis of the modified polyhedrin protein, the deleted amino acid sequence may be an amino acid sequence region at one continuous location, or amino acid sequence regions at two or more separated locations.

The wild-type polyhedrin protein that is the basis of the modified polyhedrin protein of the present embodiment is not particularly limited as long as it is a polyhedrin protein that forms a polyhedron, and may be, for example, a protein including an amino acid sequence set forth in SEQ ID NO:1.

A more specific example of the modified polyhedrin protein of the present embodiment may be, for example, a protein including an amino acid sequence set forth in SEQ ID NO:3, or a protein including an amino acid sequence in which one amino acid or a plurality of amino acids have been deleted, substituted, or added in the amino acid sequence set forth in SEQ ID NO:3, the protein having a polyhedron-forming ability.

The amino acid sequence set forth in SEQ ID NO:3 is a sequence in which 38 residues including from the 67th alanine residue to the 104th alanine residue have been deleted from the amino acid sequence set forth in SEQ ID NO:1.

### Seventh Embodiment

The above-mentioned crystalline protein may be a fusion protein in which a target peptide has been added between the 66th and 67th amino acids of the amino acid sequence set forth in SEQ ID NO:3, the fusion protein having a polyhedron-forming ability; or a fusion protein including an amino acid sequence in which one amino acid or a plurality of amino acids have been deleted, substituted, or added in the above-described fusion protein, the fusion protein having a polyhedron-forming ability.

The fusion protein of the present embodiment is a fusion protein of the above-mentioned modified polyhedrin protein and a target peptide. Furthermore, in the fusion protein of the present embodiment, the target peptide is added in the region where some amino acids have been deleted from the wild-type polyhedrin protein that is the basis of the modified polyhedrin protein.

On the inside of a polyhedron obtained by crystallizing the fusion protein of the present embodiment, target peptides are regularly disclosed. Therefore, a crystal structure analysis of the target peptide can be easily carried out using the fusion protein of the present embodiment.

### Eighth Embodiment

The above-mentioned crystalline protein may be a protein that has a site including an amino acid having a property of coordinating to a metal atom and has a polyhedron-forming ability, and the target molecule may be a substance containing a metal atom. The site including an amino acid having a property of coordinating to a metal atom may be located at the C-terminus of the above-mentioned protein.

Examples of the amino acid having a property of coordinating to a metal atom include histidine, glutamic acid, aspartic acid, cysteine, and serine. The site including an amino acid having a property of coordinating to a metal atom may be a site including one or more amino acids selected from the above-mentioned amino acids, and examples include a site in which about six histidines are contiguously arranged; a site where an amino acid having a property of coordinating to a metal atom is exposed in the space inside a crystal of a polyhedron; and a site where an amino acid having a property of coordinating to a metal atom is exposed on the crystal surface of a polyhedron.

### Ninth Embodiment

The cell-free protein synthesis system may include a target molecule having a molecular weight of 10 to 100,000.

By causing the above-mentioned crystalline protein to be expressed in the cell-free protein synthesis system including a target molecule, the target molecule can be enclosed in a crystal of the above-mentioned crystalline protein.

As described above, the target molecule can also be bound to the surface of a polyhedron by modifying the amino acid sequence of the protein having a polyhedron-forming ability.

### (Target Molecule)

The molecular weight of the target molecule is preferably 10 to 100,000, more preferably 10 to 60,000, and even more preferably 10 to 30,000. When the molecular weight of the target molecule is within the above-mentioned range, the target molecule is likely to be enclosed in the polyhedron crystal.

The target molecule is not particularly limited, and examples thereof include an inorganic substance, an organic substance, and a substance containing a metal atom. The organic substance may be, for example, a peptide, a protein, fluorescein, or the like.

Examples of the substance containing a metal atom include a metal complex, a metal atom-containing protein, and a metal atom-containing organic compound.

The metal complex may be a substance containing one or more metal atoms selected from copper, nickel, zinc, cobalt, manganese, iron, ruthenium, rhenium, and the like, and examples thereof include Mn(CO)5Br, Mn(CO)5Cl, Ru2(CO)6Cl4, Fe3(CO)12, and Re(CO)5Cl.

Examples of the metal atom-containing protein include cytochrome P450, heme proteins typified by hemoglobin, superoxide dismutase (SOD), and non-heme proteins typified by alcohol dehydrogenase.

Examples of the metal atom-containing organic compound include protoporphyrin and chlorophyll.

### [Fusion Protein]

According to an embodiment, the present invention provides a fusion protein in which an amino acid sequence of the 70th amino acid to the 77th amino acid of the amino acid sequence set forth in SEQ ID NO: 1 has been substituted with a target peptide, the fusion protein having a polyhedron-forming ability, or a fusion protein including an amino acid sequence in which one amino acid or a plurality of amino acids have been deleted, substituted, or added in the above-described fusion protein, the fusion protein having a polyhedron-forming ability.

The fusion protein of the present embodiment is the same as the fusion protein described above in the third embodiment of the method for producing a crystal.

A crystal structure analysis of the target peptide can be easily performed by analyzing the crystal structure of polyhedra obtained by crystallizing the fusion protein of the present embodiment.

Furthermore, the size of the polyhedra crystals can be controlled by appropriately designing the amino acid sequence of the target peptide.

### [Polyhedron]

According to an embodiment, the present invention provides a polyhedron crystal in which the fusion protein described above in [Fusion Protein] has been crystallized. When the fusion protein of the present embodiment is expressed in a cell-free protein synthesis system as described above or in a cell, the fusion protein forms a polyhedron crystal.

### [Polyhedron-Target Molecule Complex]

According to an embodiment, the present invention provides a polyhedron-target molecule complex in which a target molecule is enclosed in the crystal structure of the polyhedron described above in [Polyhedron]. When a polyhedron crystal is formed from the above-mentioned fusion protein, by bringing the target molecule into contact with the fusion protein, a polyhedron-target molecule complex in which the target molecule is enclosed in the crystal structure of the polyhedron can be obtained.

### [Nucleic Acid]

According to an embodiment, the present invention provides a nucleic acid encoding the fusion protein described above in [Fusion Protein]. According to the present embodiment, the nucleic acid may be RNA or may be DNA.

### [Kit for Producing Cell-Free System Crystal]

According to an embodiment, the present invention provides a kit for producing a cell-free system crystal, the kit including: a nucleic acid encoding a crystalline protein; and a reagent for a cell-free protein synthesis system.

A cell-free protein synthesis system can be conveniently prepared by using the reagent attached to the kit of the present embodiment. Examples of the cell-free protein synthesis system include those described above in [Production method]. The cell-free protein synthesis system may be a synthesis system that utilizes a cell extract or may be a synthesis system in which factors required for translation have been reconstituted.

When the cell-free protein synthesis system is a synthesis system that utilizes a cell extract, the kit may include a cell extract as reagent for the cell-free protein synthesis system attached to the kit. The biological species of the cells from which the cell extract is derived is not particularly limited, and the above-mentioned ones in [Production method] may be mentioned.

When the cell-free protein synthesis system is a synthesis system in which the factors required for translation have been reconstituted, the kit may include the factors required for translation. The biological species from which the factors required for translation are derived is not particularly limited.

The kit of the present embodiment may include factors that increase the efficiency of protein synthesis, such as amino acids and energy molecules. Examples of the factors for increasing the efficiency of protein synthesis include the ones mentioned above in

### [Production Method].

The nucleic acid included in the kit of the present embodiment may be RNA or may be DNA. The sequence of the above-mentioned nucleic acid encoding the protein may be synonymously substituted such that the expression of the protein is optimized in the cell-free protein synthesis system.

When the nucleic acid is DNA, the DNA may be linear or may be circular. A circular DNA may be, for example, an expression vector such as a plasmid. A plasmid may have a promoter upstream of the above-mentioned DNA sequence encoding the protein or may have a ribosome-binding site.

The kit of the present embodiment may include factors required for transcription. Examples of the factors required for transcription include the factors described above in [Production method]. When the nucleic acid encoding the protein is DNA, RNA can be synthesized by transcription using the factors required for transcription that are included in the kit of the present embodiment.

### First Embodiment

According to the kit of the present embodiment, the crystalline protein may be a protein according to any one of the following (A) to (C):
(A) a cytoplasmic polyhedral protein, a nuclear polyhedral protein, an insecticidal protein, cathepsin B, luciferase, reovirus nonstructural protein (μNS), CipA, or fusolin protein (Fusolin);
(B) a protein including an amino acid sequence in which one amino acid or a plurality of amino acids have been deleted, substituted, or added in an amino acid sequence of the protein of (A), the protein having a crystal-forming ability; and
(C) a fusion protein of the protein of (A) or (B) and a target peptide.

For example, by adding a nucleic acid encoding the above-mentioned crystalline protein attached to the kit of the present embodiment, to the cell-free protein synthesis system attached to the kit of the present embodiment, the crystalline protein is synthesized, and thereby protein crystals can be easily produced.

### Second Embodiment

According to the kit of the present embodiment, the crystalline protein may be one selected from the group consisting of the following (i), (ii), (iii), and (iv):
(i) a protein including an amino acid sequence set forth in SEQ ID NO:1, or a protein including an amino acid sequence in which one amino acid or a plurality of amino acids have been deleted, substituted, or added in the amino acid sequence set forth in SEQ ID NO:1, the protein having a polyhedron-forming ability;
(ii) a protein including an amino acid sequence set forth in SEQ ID NO:2, or a protein including an amino acid sequence in which one amino acid or a plurality of amino acids have been deleted, substituted, or added in the amino acid sequence set forth in SEQ ID NO:2, the protein having a polyhedron-forming ability;
(iii) a protein including an amino acid sequence set forth in SEQ ID NO: 18, or a protein including an amino acid sequence in which one amino acid or a plurality of amino acids have been deleted, substituted, or added in the amino acid sequence set forth in SEQ ID NO: 18, the protein having a crystal-forming ability; and
(iv) a fusion protein in which an amino acid sequence of the 70th amino acid to the 77th amino acid of the amino acid sequence set forth in SEQ ID NO: 1 has been substituted with a target peptide, the fusion protein having a polyhedron-forming ability, or a protein including an amino acid sequence in which one amino acid or a plurality of amino acids have been deleted, substituted, or added in the fusion protein, the protein having a polyhedron-forming ability.

For example, by adding a nucleic acid encoding the above-mentioned crystalline protein attached to the kit of the present embodiment, to the cell-free protein synthesis system attached to the kit of the present embodiment, the crystalline protein is synthesized, and thereby protein crystals can be easily produced.

### Third Embodiment

When the kit of the present embodiment includes a nucleic acid encoding the above-mentioned item (i) or (iv), the kit of the present embodiment may further include a nucleic acid encoding a fusion protein in which a target peptide is bonded to the C-terminus of an amino acid sequence set forth in SEQ ID NO:4 or to the C-terminus of an amino acid sequence in which one amino acid or a plurality of amino acids have been deleted, substituted, or added in the amino acid sequence set forth in SEQ ID NO:4.

By adding the nucleic acid attached to the present embodiment to the cell-free protein synthesis system attached to the kit of the present embodiment, a protein having a polyhedron-forming ability and a fusion protein are expressed, these form a polyhedron crystal, and the fusion protein can form a portion of the polyhedron crystal.

Polyhedra crystals produced by the above-mentioned production method can be collected by, for example, centrifuging the reaction solution of the translation reaction.

The polyhedra crystals can be stored in distilled water or in a solution such as a buffered saline solution or distilled water including antibiotics.

Since the target molecule enclosed in the polyhedron is protected by the polyhedron, by enclosing the target molecule in a polyhedron-target molecule complex, stability against ultraviolet radiation, heat, drying, immersion in a urea solution, immersion in an acid, immersion in a solution including a surfactant, and the like can be enhanced.

Furthermore, since the polyhedron-target molecule complex has cell affinity (low biotoxicity), the polyhedron-target molecule complex can be applied to the storage and controlled release of a metal-containing drug and the control of reactions of small molecules in vivo.

The control of reactions of small molecules includes capture of small molecules, release of small molecules, synthesis of small molecules, breakdown of small molecules, and the like.

Furthermore, for example, the target molecule can be released in a controlled manner by changing the pH of the solution including the polyhedron to be, for example, 10 or higher. In addition to changes in the pH, by enclosing a target molecule that releases an active substance by light irradiation, temperature changes, and addition of active molecules, in the complex, controlled release of the active substance by light irradiation, temperature changes, and addition of active molecules is enabled. Examples of the target molecule that releases a substance by light irradiation include Mn(CO)5Br. Mn(CO)5Br releases CO by light irradiation.

By the way, it is known that gas molecules such as CO, NO, and O2 are greatly involved in cerebral infarction, Alzheimer's disease, carcinogenesis, and liver diseases. These gas molecules infiltrate into cells and affect neurotransmission, transcription factor activity, and the like.

Conventionally, it has been reported that CO-releasing materials were supported on polymers, gels, or porous materials, and release of CO at normal temperature and normal pressure and release of CO in water were performed; however, there is a problem that the materials are biotoxic and the synthesis process is complicated. In contrast, the polyhedron-target molecule complex has cell affinity and is easily produced.

For example, a new material having cell affinity and capable of controlled release of gas molecules can be provided by polyhedra that enclose materials for releasing gas molecules such as CO, NO, or 02 as target molecules. By incorporating such a material into, for example, an evaluation system established by iPS cells or ES cells, the material can be utilized for the development of pharmaceutical products for cerebral infarction, Alzheimer's disease, carcinogenesis, liver diseases, and the like.

According to the present invention, as described above, polyhedra can be crystallized by causing polyhedrin protein to be expressed in an extremely small amount, such as about 50 μL, of the reaction solution. Therefore, operations including from expression to crystallization of polyhedrin protein can be carried out in the wells of 24-wells, 96-wells, and 384-wells.

Since a large number of samples on a small scale such as described above can be simultaneously handled, it is possible to verify the effect of a drug with high throughput by using crystallized polyhedra. For example, it becomes possible to efficiently screen active drugs by enclosing a target substance in polyhedra and allowing a library of drug candidates to act on the target substance enclosed in the polyhedra under appropriate conditions.

### EXAMPLES

Hereinafter, the present invention will be described by way of Examples; however, the present invention is not intended to be limited to the following Examples.

### Experimental Example 1

### (Crystallization of Wild-Type Polyhedrin by Cell-Free Protein Synthesis System)

A wheat germ cell-free protein synthesis system was used to express wild-type polyhedrin of CPV, and polyhedra crystals were formed.

For the wheat germ cell-free protein synthesis system, SUB-AMIX SGC, WEPRO 9240, and pEU-E01-MCS vectors manufactured by CellFree Sciences Co., Ltd. were used.

First, a cDNA encoding the wild-type polyhedrin protein set forth in SEQ ID NO: 1 was added into the pEU-E01-MCS vector, and an expression vector was obtained. 2 μg of the obtained expression vector was prepared, this was added to a Transcription Premix LM tube and then incubated at 37° C. for 6 hours to synthesize mRNA, and a transcription reaction solution was obtained.

The transcription reaction solution was placed on the bottom of wells containing the SUB-AMIX SGC to form a layer in the wells. Subsequently, the mixture was kept warm at 15° C. for 20 hours, a translation reaction was carried out, and wild-type polyhedrin was synthesized.

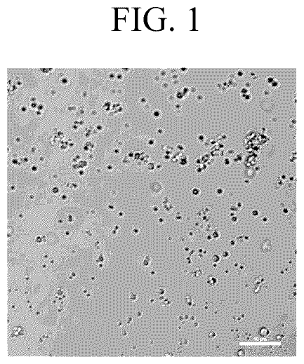

One day after the initiation of the translation reaction, crystals formed at the bottom of the wells were observed. The results are shown in FIG. 1. FIG. 1 is a photograph of wild-type polyhedrin crystals obtained by a cell-free protein synthesis system. As a result, it was found that crystals are obtained in just one day by means of a cell-free protein synthesis system, by a simple operation as described above.

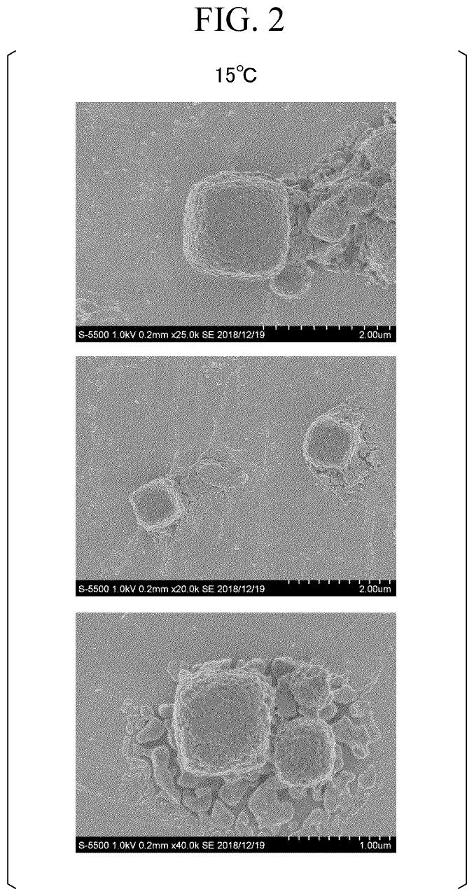

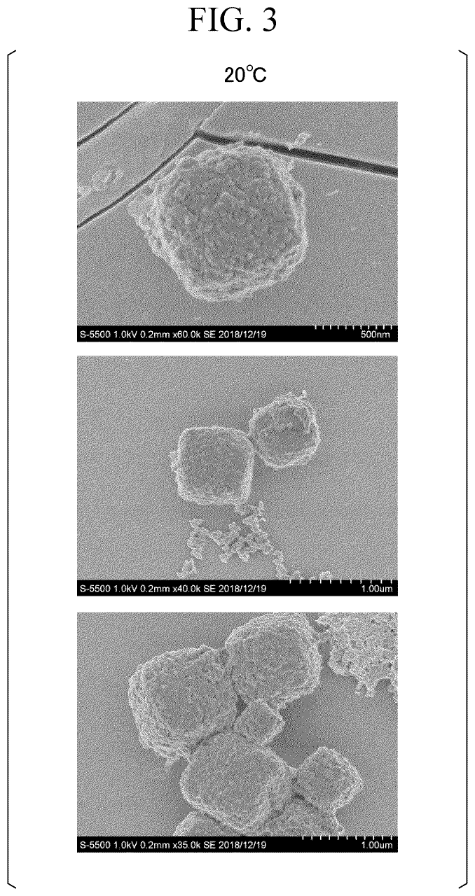

Subsequently, the obtained crystals were observed by scanning electron microscopy (SEM). The results are shown in FIG. 2. Furthermore, crystals were similarly produced by changing the temperature at the time of the translation reaction to 20° C., and the obtained crystals were observed by SEM. The results are shown in FIG. 3.

As a result, the size of the crystals obtained by performing the translation reaction at 15° C. and 20° C. was 0.5 μm to 1.5 μm. Furthermore, no significant difference in the size and shape of the crystals was recognized between the case where the translation reaction was carried out at 15° C. and the case where the translation reaction was carried out at 20° C.

Regarding a method for crystallizing a protein, a method of purifying the protein from cells and then performing crystallization is generally used. According to such a method, a duration of two weeks or longer was required in order to obtain crystals, and a lot of effort was also required.

Furthermore, in order to produce polyhedra crystals, a method of infecting cells with a baculovirus to produce polyhedra crystals in the cells may be employed. In this case, a duration of about 2 weeks was required to prepare the baculovirus.

On the other hand, according to the method for producing a crystal of the present invention, as illustrated in Experimental Example 1, it was found that crystallization can be achieved in a short time of about one day by a very simple method.

### Experimental Example 2

### (X-Ray Structural Analysis of Crystal Obtained by Cell-Free Protein Synthesis System)

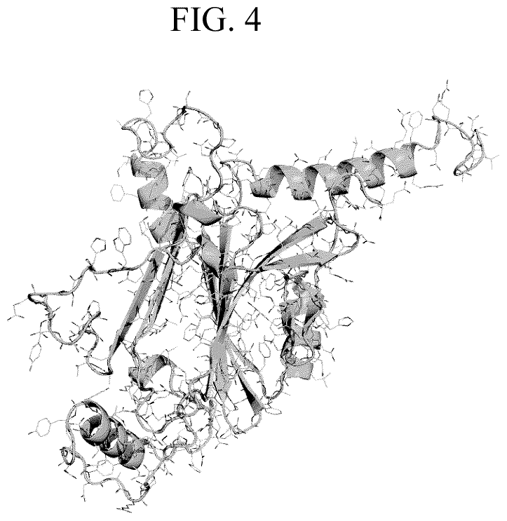

For the wild-type polyhedrin crystal of CPV obtained in Experimental Example 1, the three-dimensional structure was analyzed by X-ray structural analysis. The resolution was set to 2.5 Å. The results are shown in FIG. 4.

As a result, the three-dimensional structure of the wild-type polyhedrin crystal obtained by the cell-free protein synthesis system was not significantly different from the three-dimensional structure of the wild-type polyhedrin crystal synthesized by a conventional method, that is, intracellularly. Furthermore, even when the resolution was set to 1.70 Å, the three-dimensional structure could be analyzed.

### Experimental Example 3

### (Crystallization Conditions for Crystal Obtained by Cell-Free Protein Synthesis System)

The amount of solution from which wild-type polyhedrin crystals of CPV having a well-regulated shape were obtained was investigated by adjusting the amount of the solution after the translation reaction obtained in Experimental Example 1.

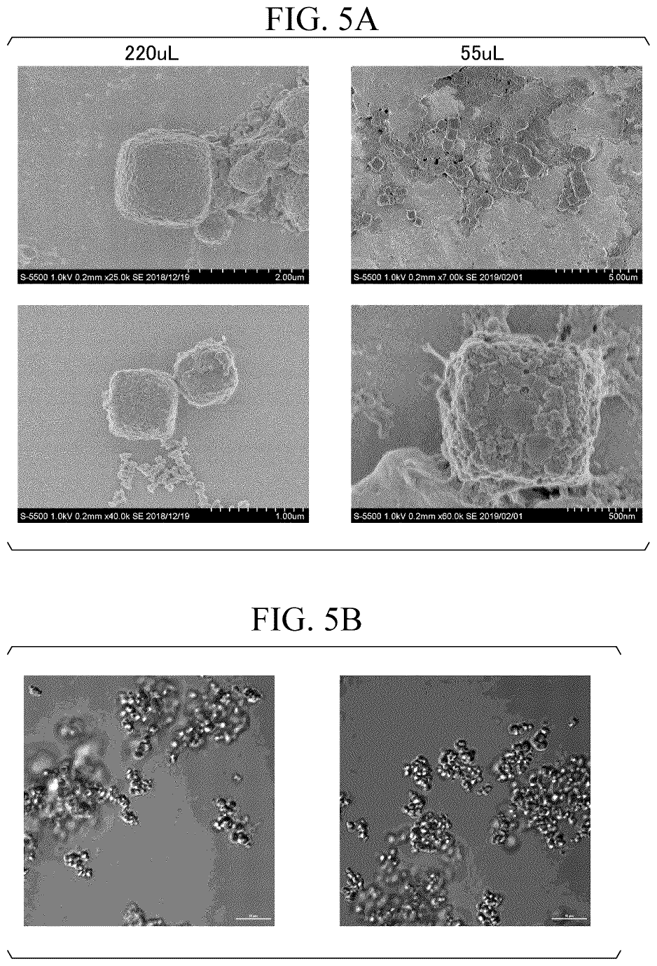

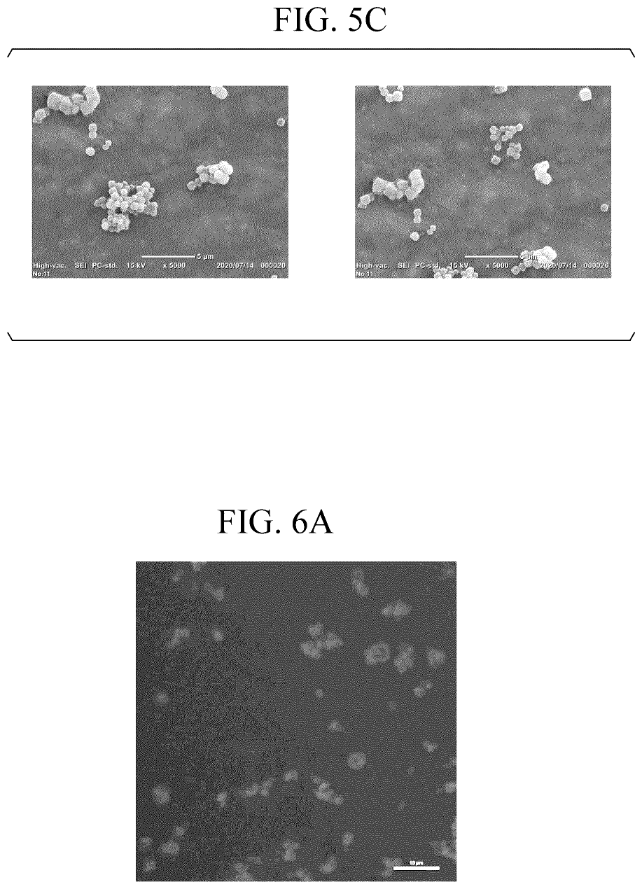

Specifically, the morphology of each crystal obtained from solutions (220 μL, 55 μL, 22 μL) after the translation reaction as obtained in Experimental Example 1 was observed by optical microscopy or SEM. The results are shown in FIG. 5A to FIG. 5C. FIG. 5A is SEM images of crystals obtained from 220 μL of the solution and 55 μL of the solution. FIG. 5B is optical microscopic images of crystals obtained from 22 μL of the solution, and FIG. 5C is SEM images of crystals obtained from 22 μL of the solution.

As a result, crystals having a well-regulated cubic shape were obtained from 220 μL of the solution after the translation reaction. Furthermore, crystals having a cubic shape were also obtained from 55 μL of the solution and 22 μL of the solution after the translation reaction.

### Experimental Example 4

### (Crystallization of Polyhedrin Fragment-GFP Fusion Protein)

A fusion protein obtained by fusing a fragment of polyhedrin protein of CPV with -GFP, and wild-type polyhedrin protein of CPV were co-expressed by a cell-free protein synthesis system to prepare polyhedra crystals enclosing GFP.

cDNA encoding the GFP fusion protein set forth in SEQ ID NO:5 and cDNA encoding the wild-type polyhedrin protein set forth in SEQ ID NO: 1 were each added into a pEU-E01-MCS vector in the same manner as in Experimental Example 1, and expression vectors were produced.

Crystals were produced through expression by a cell-free protein synthesis system in the same manner as in Experimental Example 1. A fusion protein set forth in SEQ ID NO: 5 is a protein in which GFP protein is fused to the C-terminal side of the H1 α-helix located at the N-terminus of wild-type polyhedrin protein.

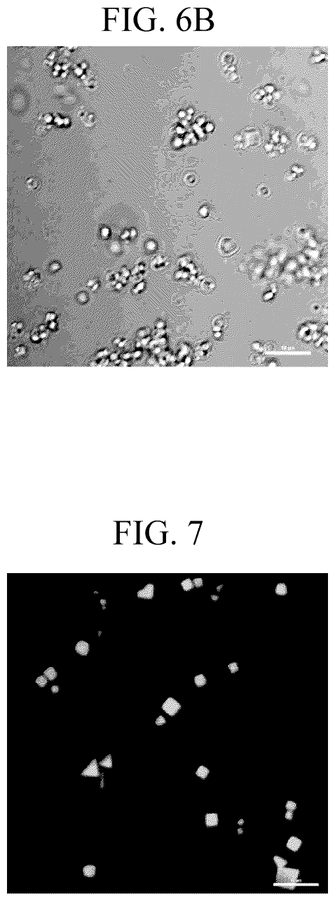

Crystals formed in the solution after the translation reaction were observed. The results are shown in FIG. 6. FIG. 6A is a fluorescence image of GFP taken by irradiating crystals with excitation light. FIG. 6B is the result of superimposing a bright field image and a fluorescence image of the crystals.

As a result, it was found that polyhedra crystals enclosing GFP were obtained.

### Experimental Example 5

### (Crystallization of Polyhedron Enclosing Fluorescein)

Crystals of a polyhedron enclosing fluorescein were produced by causing the wild-type polyhedrin protein of CPV to be expressed by a cell-free protein synthesis system including fluorescein.

The expression vector expressing the wild-type polyhedrin protein, which was obtained in Experimental Example 1, and fluorescein were added to the cell-free protein synthesis system to express the wild-type polyhedrin protein. The obtained crystals were observed. The results are shown in FIG. 7.

FIG. 7 is a fluorescence image taken by irradiating polyhedra crystals enclosing fluorescein with excitation light. As a result, it was found that polyhedra crystals enclosing fluorescein were obtained.

### Experimental Example 6

### (Crystallization of Mutant Polyhedrin Protein)

Four kinds of mutant polyhedrin proteins in which the amino acid sequence of L1 or a portion of L1 of the wild-type polyhedrin protein of CPV had been substituted with the following peptides, were expressed by a cell-free protein synthesis system to produce crystals. Furthermore, crystals were also produced inside capillary tubes using a smaller amount of a mutant polyhedrin protein solution.

The 70th to 77th residues of the wild-type polyhedrin protein including the amino acid sequence set forth in SEQ ID NO: 1 are referred to as L1 (loop region) and including an amino acid sequence set forth in SEQ ID NO:6. The amino acid sequence in the vicinity of L1 was substituted as follows with peptides or the like as shown below.

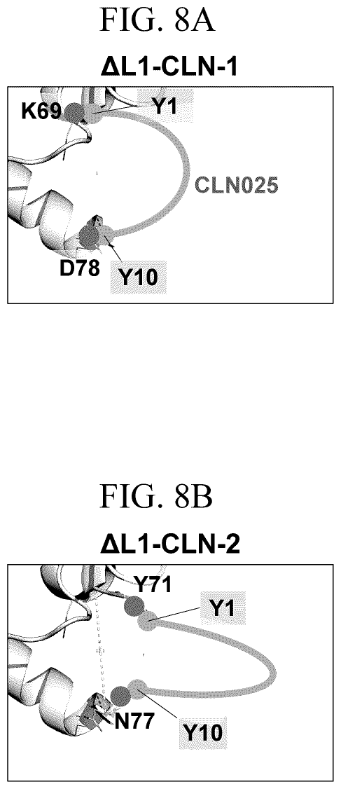

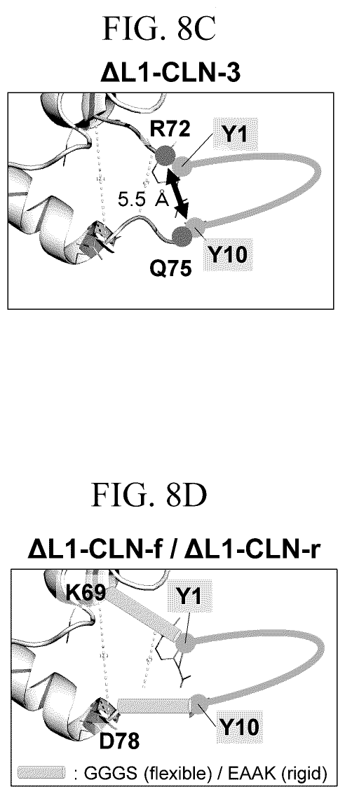

An amino acid sequence of the 70th to 77th residues of the wild-type polyhedrin protein set forth in SEQ ID NO: 1 was substituted with CLN025 peptide set forth in SEQ ID NO:7, and a mutant polyhedrin protein (ΔL1-CLN-1) set forth in SEQ ID NO:8 was designed. A schematic diagram of the three-dimensional structure of this mutant polyhedrin protein in the vicinity of the CLN025 peptide is shown in FIG. 8A.

A mutant polyhedrin protein (ΔL1-CLN-2) set forth in SEQ ID NO:9 was designed, in which two amino acid residues were added on the N-terminal side of the target peptide, and one amino acid residue was added on the C-terminal side of the target peptide in the mutant polyhedrin protein set forth in SEQ ID NO:8.

It can be said that the amino acid sequence of ΔL1-CLN-2 is an amino acid sequence obtained by substituting an amino acid sequence of the 72nd to 76th residues of the wild-type polyhedrin protein set forth in SEQ ID NO: 1 with the CLN025 peptide set forth in SEQ ID NO:7.

A schematic diagram of the three-dimensional structure of this mutant polyhedrin protein in the vicinity of the CLN025 peptide is shown in FIG. 8B.

A mutant polyhedrin protein (ΔL1-CLN-3) set forth in SEQ ID NO: 10 was designed, in which three amino acid residues were added on the N-terminal side of the target peptide, and three amino acid residues were added on the C-terminal side of the target peptide in the mutant polyhedrin protein set forth in SEQ ID NO:8.

It can be said that the amino acid sequence of ΔL1-CLN-3 is an amino acid sequence obtained by substituting an amino acid sequence of the 73rd and 74th residues of the wild-type polyhedrin protein set forth in SEQ ID NO:1 with the CLN025 peptide set forth in SEQ ID NO:7.

A schematic diagram of the three-dimensional structure of this mutant polyhedrin protein in the vicinity of the CLN025 peptide is shown in FIG. 8C.

A mutant polyhedrin protein (ΔL1-CLN-f) set forth in SEQ ID NO:12 was designed, in which four amino acid residues were added on the N-terminal side of the target peptide, and four amino acid residues were added on the C-terminal side of the target peptide in the mutant polyhedrin protein set forth in SEQ ID NO:8. A 4-residue peptide set forth in SEQ ID NO: 13 is linked to the N-terminus and the C-terminus of CLN025 peptide. The 4-residue peptide set forth in SEQ ID NO: 13 has a high degree of freedom in the three-dimensional structure.

It can be said that the amino acid sequence of ΔL1-CLN-f is an amino acid sequence obtained by substituting an amino acid sequence of the 70th to 77th residues of the wild-type polyhedrin protein set forth in SEQ ID NO: 1 with a peptide including an amino acid sequence set forth in SEQ ID NO:11.

A schematic diagram of the three-dimensional structure of this mutant polyhedrin protein near the CLN025 peptide is shown in FIG. 8D.

A mutant polyhedrin protein (ΔL1-CLN-r) set forth in SEQ ID NO: 15 was designed, in which four amino acid residues were added on the N-terminal side of the target peptide, and four amino acid residues were added on the C-terminal side of the target peptide in the mutant polyhedrin protein set forth in SEQ ID NO:8. A 4-residue peptide set forth in SEQ ID NO: 16 is linked to the N-terminus and the C-terminus of CLN025 peptide. The 4-residue peptide set forth in SEQ ID NO:16 has a low degree of freedom in the three-dimensional structure.

It can be said that the amino acid sequence of ΔL1-CLN-r is an amino acid sequence obtained by substituting an amino acid sequence of the 70th to 77th residues of the wild-type polyhedrin protein set forth in SEQ ID NO: 1 with a peptide including an amino acid sequence set forth in SEQ ID NO: 14.

A schematic diagram of the three-dimensional structure of this mutant polyhedrin protein near the CLN025 peptide is shown in FIG. 8D.

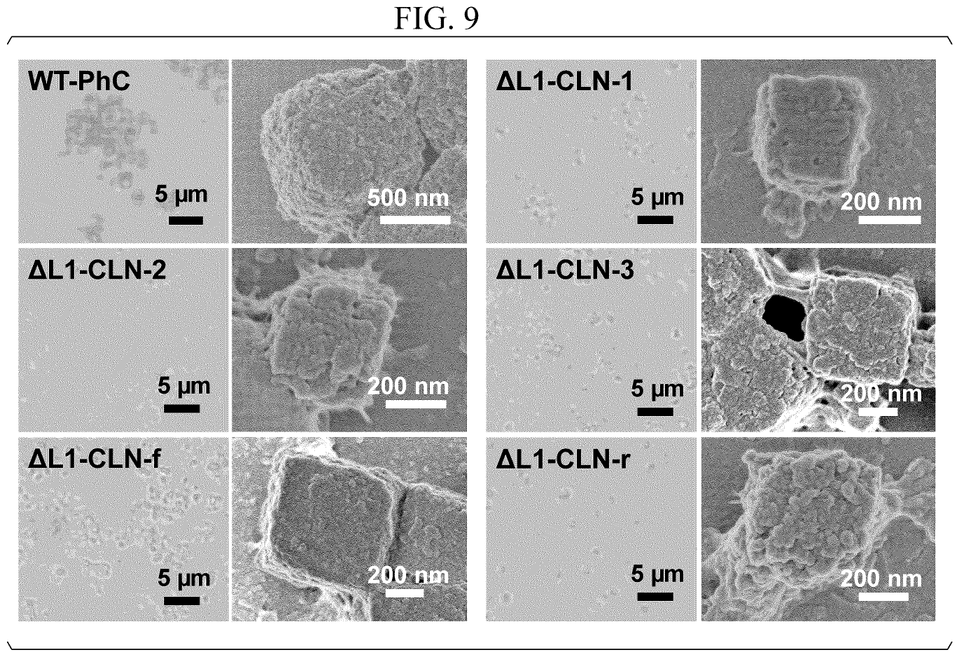

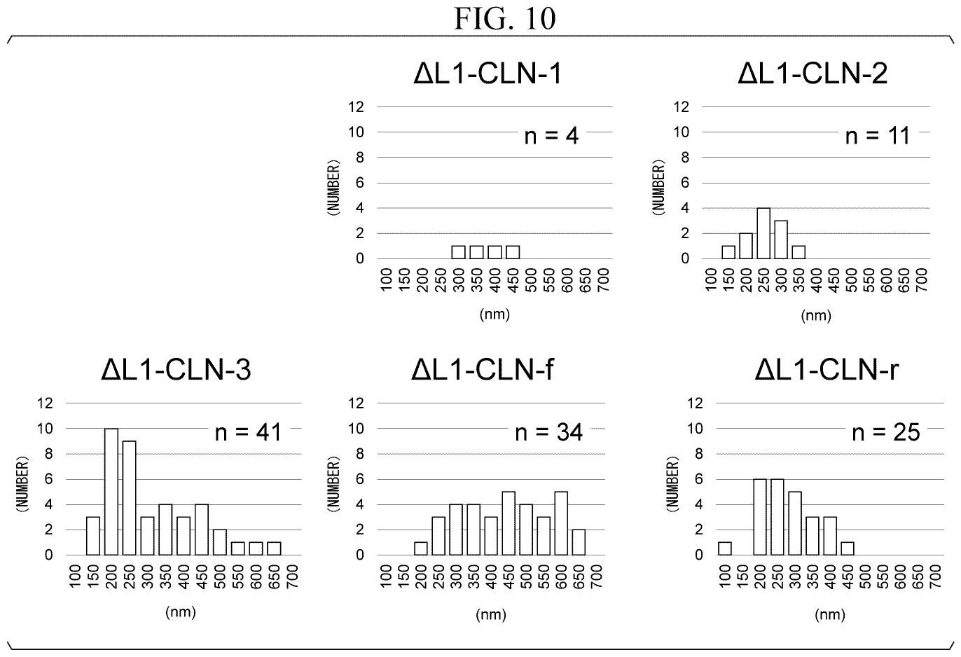

Each of ΔL1-CLN-1, ΔL1-CLN-2, ΔL1-CLN-3, ΔL1-CLN-f, and ΔL1-CLN-r was expressed by a cell-free protein synthesis system in the same manner as in Experimental Example 1 to produce crystals, and the obtained crystals were observed. The results are shown in FIG. 9 and FIG. 10.

FIG. 9 is images taken by optical microscopy and images taken by SEM of the obtained crystals. As a result, crystals showing a cubic shape were obtained for all the mutant polyhedrin proteins.

FIG. 10 is graphs obtained by measuring the length of one side of the obtained cubic crystals and showing the results as histograms. The average value of the length of one side of the crystals was 500 nm or less.

Furthermore, Δ3, which is a mutant polyhedrin protein having three amino acid residues (G192, S193, A194) deleted, as set forth in SEQ ID NO: 17, was expressed in a cell-free protein synthesis system. 18 μL of the solution of the cell-free protein synthesis system was injected into a capillary tube, and crystals were obtained. The obtained crystals were observed using a microscope.

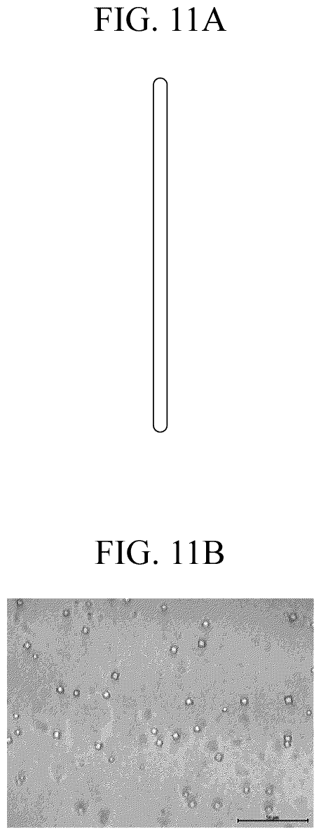

FIG. 11A is a photograph of the capillary tube used, and FIG. 11B is a bright field photographic image of the obtained crystals. When crystals are produced using a capillary tube, the crystals can be produced from a smaller amount of the reaction solution, and the crystals in the capillary tube can be efficiently utilized.

### Experimental Example 7

### (Identification of Mutant Polyhedrin Protein)

The crystals obtained in Experimental Example 6 were analyzed by mass spectrometry and SDS-PAGE, and it was verified whether the obtained crystals were mutant polyhedrin proteins. Furthermore, the polyhedra crystals obtained by expressing wild-type polyhedrin protein (WT-PhC) in Sf9 cells were also analyzed as a control.

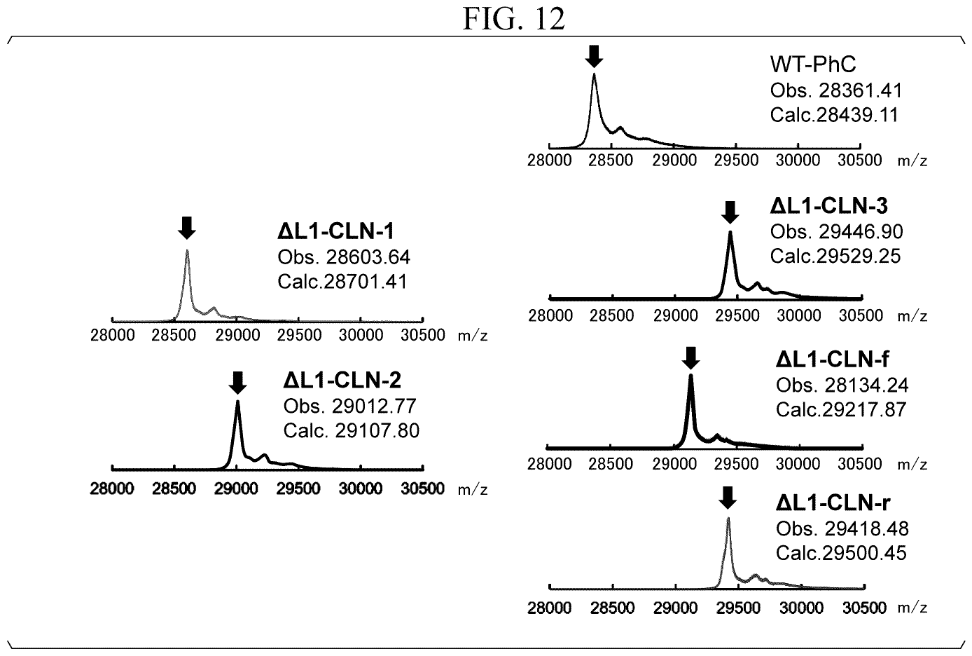

In mass spectrometry, the obtained crystals were analyzed by MALDI-TOF-MS. The results of mass spectrometry are shown in FIG. 12. In FIG. 12, Calc. represents the molecular weight calculated from the amino acid sequence, and Obs. indicates the mass estimated from the results of mass spectrometry. As a result, it was found that each of the obtained crystals was the mutant polyhedrin protein shown in Experimental Example 6.

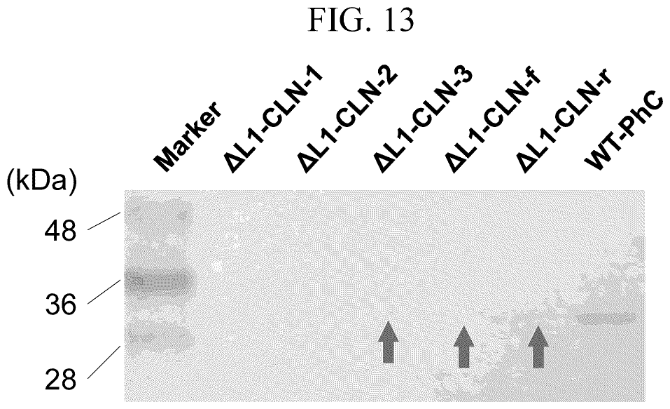

In SDS-PAGE, the obtained crystals were solubilized and subjected to electrophoresis. The results of SDS-PAGE are shown in FIG. 13. As a result, it was found that each of the obtained crystals was the mutant polyhedrin protein shown in Experimental Example 6.

### Experimental Example 8

### (X-Ray Diffraction of Mutant Polyhedrin Protein)

The crystals obtained in Experimental Example 6 were irradiated with X-rays, and X-ray diffraction was analyzed.

The solution after the translation reaction including the polyhedra crystals in Experimental Example 6 was centrifuged, and the settled crystals were introduced into a 50% PBS/ethylene glycol solution. The solution including the crystals was placed on a mesh, a large number of crystals on the mesh were irradiated with X-rays, and X-ray diffraction was observed.

As a result, when the crystals of ΔL1-CLN-3 and ΔL1-CLN-f were irradiated with X-rays, the frequency of detecting X-ray diffraction was high, and for the crystals of ΔL1-CLN-1 and ΔL1-CLN-r, X-ray diffraction was not detected. For the crystals of ΔL1-CLN-f, X-ray diffraction was detected with particularly high frequency. From these results, it was found that the crystals of ΔL1-CLN-f had a high degree of orientation. It was also suggested that structural analysis methods other than X-ray structural analysis can be applied to the crystals of ΔL1-CLN-f.

### Experimental Example 9

### (Crystallization of Cry3A Protein)

Cry3A protein set forth in SEQ ID NO:18 was expressed in the same manner as in Experimental Example 1 using a wheat germ cell-free protein synthesis system, and the obtained protein solution was maintained at 4° C. or 20° C. to form crystals. The obtained crystals were observed by optical microscopy or scanning electron microscopy.

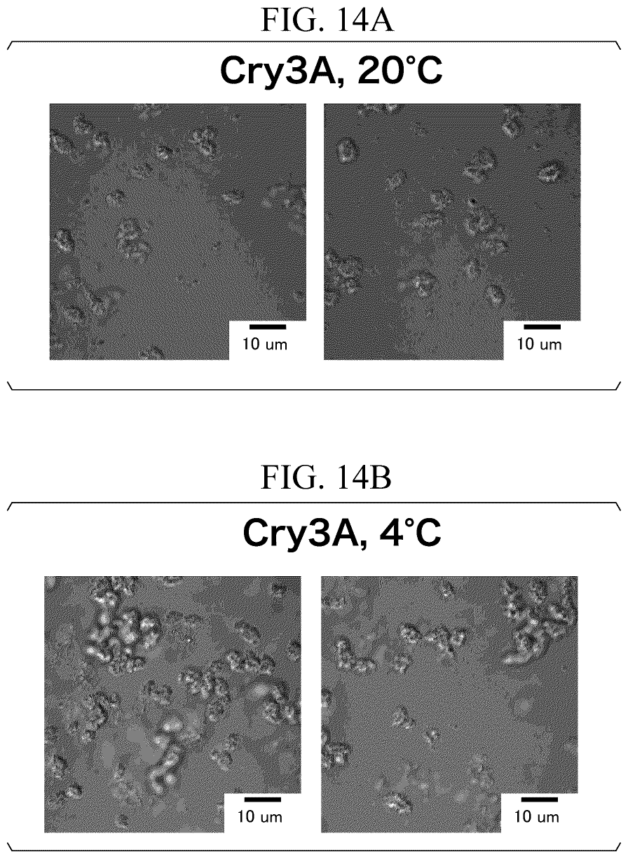

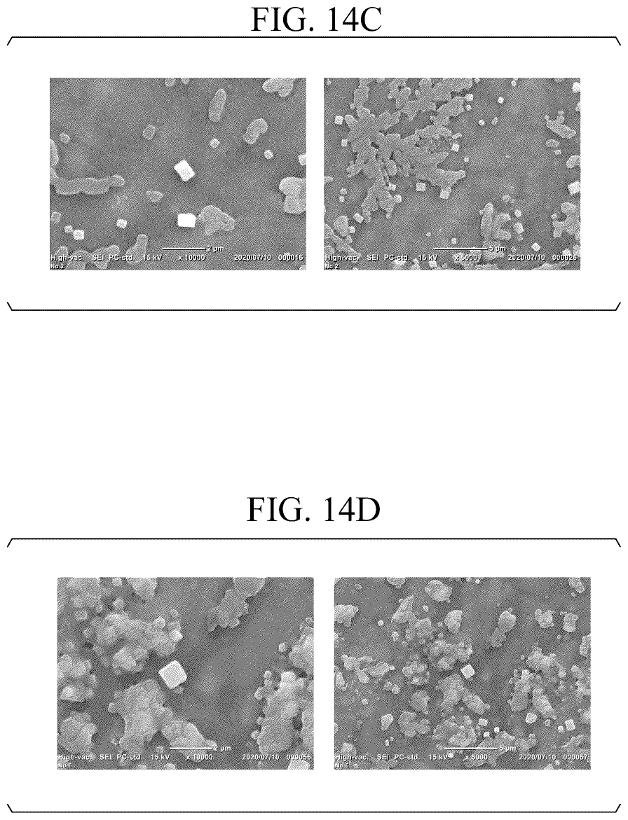

FIG. 14A is optical microscopic images of crystals produced at 20° C., and FIG. 14B is optical microscopic images of crystals produced at 4° C. FIG. 14C is SEM images of crystals produced at 20° C., and FIG. 14D is SEM images of crystals produced at 4° C. It was found that crystals of Cry3A were obtained in the same manner as in the case of the above-mentioned polyhedrin protein in Experimental Example 1.

### Experimental Example 10

### (Crystallization of μNS Protein)

μNS protein set forth in SEQ ID NO:22 was expressed in the same manner as in Experimental Example 1 using a wheat germ cell-free protein synthesis system, and the obtained protein solution was maintained at 4° C. or 20° C. to form crystals. The obtained crystals were observed by optical microscopy or electron microscopy.

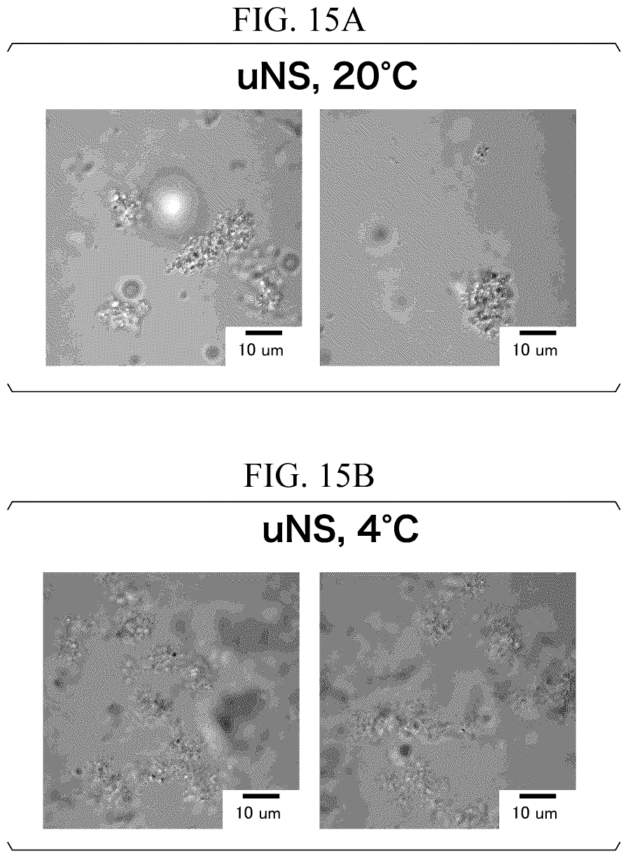

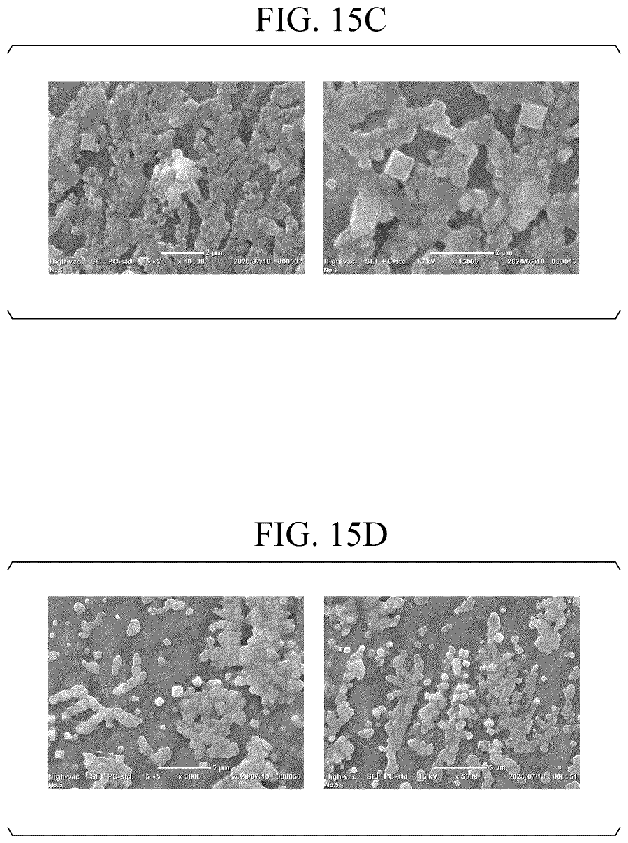

FIG. 15A is optical microscopic images of crystals produced at 20° C., and FIG. 15B is optical microscopic images of crystals produced at 4° C. FIG. 15C is SEM images of crystals produced at 20° C., and FIG. 15D is SEM images of crystals produced at 4° C. It was found that crystals of μNS protein were obtained in the same manner as in the case of the above-mentioned polyhedrin protein in Experimental Example 1.

### Experimental Example 11

### (Crystallization of Cathepsin B Protein)

Cathepsin B protein set forth in SEQ ID NO: 19 was expressed in the same manner as in Experimental Example 1 using a wheat germ cell-free protein synthesis system, and the obtained protein solution was maintained at 4° C. or 20° C. to form crystals. The obtained crystals were observed by optical microscopy or electron microscopy.

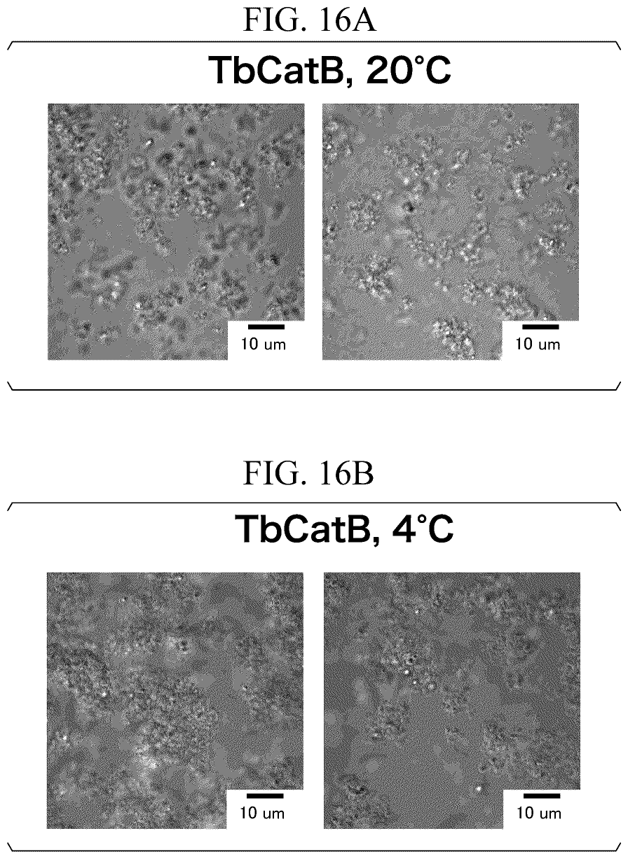

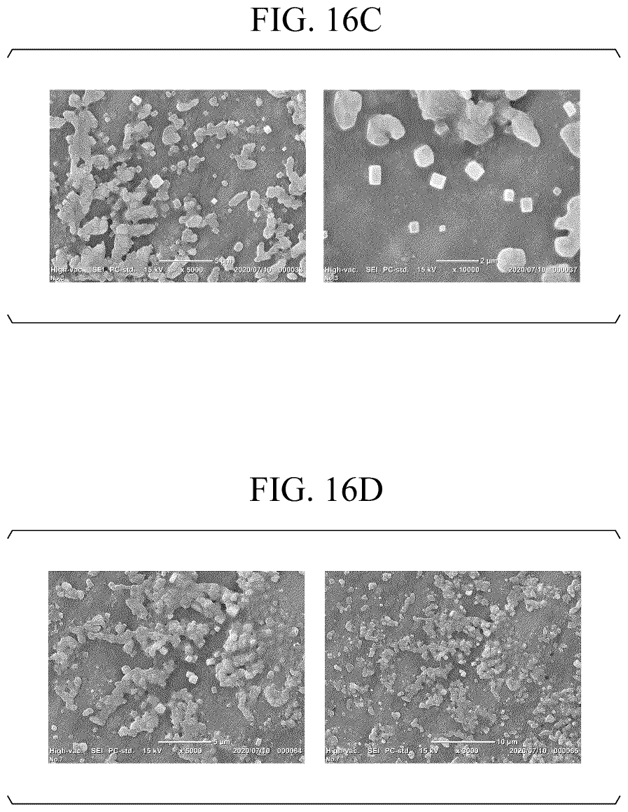

FIG. 16A is optical microscopic images of crystals produced at 20° C., and FIG. 16B is optical microscopic images of crystals produced at 4° C. FIG. 16C is SEM images of crystals produced at 20° C., and FIG. 16D is SEM images of crystals produced at 4° C. It was found that crystals of cathepsin B protein were obtained in the same manner as in the case of the above-mentioned polyhedrin protein in Experimental Example 1.

### Experimental Example 12

### (Crystallization of Nuclear Polyhedral Protein)

Nuclear polyhedral protein set forth in SEQ ID NO:2 was expressed in the same manner as in Experimental Example 1 using a wheat germ cell-free protein synthesis system, and the obtained protein solution was maintained at 4° C. or 20° C. to form crystals. The obtained crystals were observed by optical microscopy or electron microscopy.

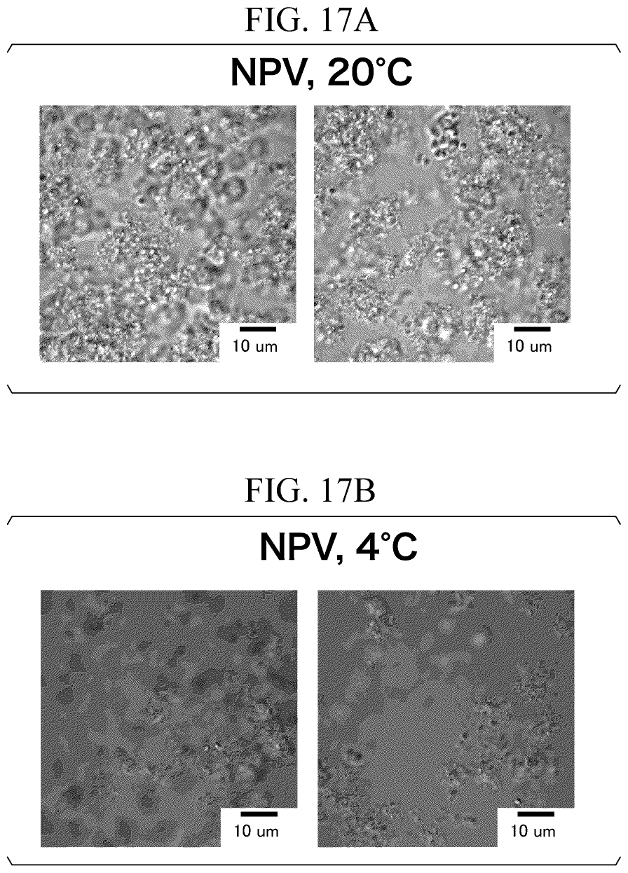

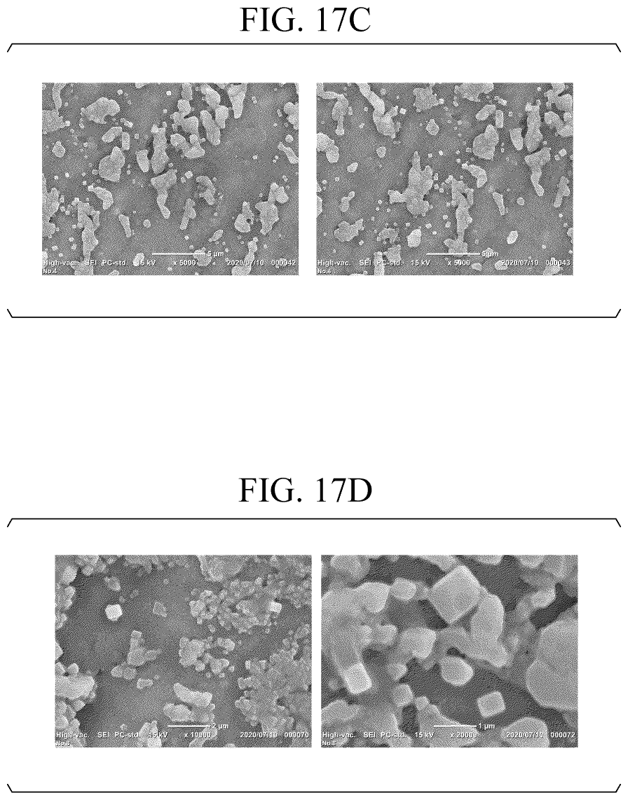

FIG. 17A is optical microscopic images of crystals produced at 20° C., and FIG. 17B is optical microscopic images of crystals produced at 4° C. FIG. 17C is SEM images of crystals produced at 20° C., and FIG. 17D is SEM images of crystals produced at 4° C. It was found that crystals of the nuclear polyhedral protein were obtained in the same manner as in the case of the above-mentioned polyhedrin protein in Experimental Example 1.

### Experimental Example 13

### (Crystallization of CipA)

CipA set forth in SEQ ID NO:24 was expressed in the same manner as in Experimental Example 1 using a wheat germ cell-free protein synthesis system, and the obtained protein solution was maintained at 20° C. to form crystals. The obtained crystals were observed by optical microscopy.

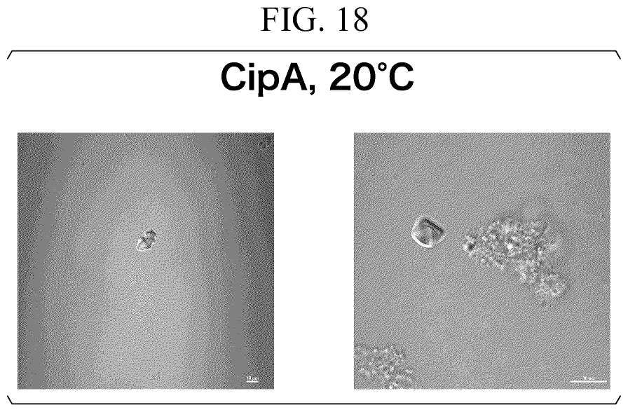

FIG. 18 is optical microscopic images of the crystals produced at 20° C. It was found that crystals of CipA were obtained in the same manner as in the case of the above-mentioned polyhedrin protein in Experimental Example 1.

### INDUSTRIAL APPLICABILITY

According to the present invention, a technology for obtaining protein crystals in a short period of time with less efforts can be provided.

What is claimed is:

1. A fusion protein comprising the amino acid sequence set forth in SEQ ID NO:1, wherein the 70th amino acid to the 77th amino acid of the amino acid sequence has been substituted with a target peptide, and/or 1 to 50 amino acids of the fusion protein have been deleted, substituted, or added, wherein the fusion protein has polyhedron-forming ability.

2. A polyhedron in which the fusion protein according to claim 1 is crystallized.

3. A polyhedron-target molecule complex, wherein a target molecule is enclosed in a crystal structure of the crystallized polyhedron according to claim 2.
